SEQUENCE LISTING (2 sequences)

SEQ ID NO: 1 - protein, 140 aa, Homo sapiens
MAGWNAYIDNLMADGTCQDAAIVGYKDSPSVWAAVPGKTFVNITPAEVGVLVGKDRSSFYVNGLTLGGQKCSVIRDSLLQDGEFSMDLRTKSTGGAPTFNVTVTKTDKTLVLLMGKEGVHGGLINKKCYEMASHLRRSQY

SEQ ID NO: 2 - DNA, 1365 bp, Homo sapiens
cccgcagggtccacacgggtcgggccgggcgcgctcccgtgcagccggctccggccccgaccgccccatgcactcccggccccggcgcaggcgcaggcgcgggcacacgcgccgccgcccgccggtccttcccttcggcggaggtgggggaaggaggagtcatcccgtttaaccctgggctccccgaactctccttaatttgctaaatttgcagcttgctaattcctcctgctttctccttccttccttcttctggctcactccctgccccgataccaaagtctggtttatattcagtgcaaattggagcaaaccctacccttcacctctctcccgccaccccccatccttctgcattgctttccatcgaactctgcaaattttgcaatagggggagggatttttaaaattgcatttgcaaagttcggtgtctgggctggcgagtgggggagggagggaatggggagtaggccccgcccctaccgtcctttgcaaataaaaatctagcggggcggggggggggaggagcaggaagtggcggtgcgagggctgctgcacagcgagcggagccgcggtccggacggcagcgcgtgccccgagctctccgcctccccccgcccgccagccgaggcagctcgagcccagtccgcggccccagcagcagcgccgagagcagccccagtagcagcgccatggccgggtggaacgcctacatcgacaacctcatggcggacgggacctgtcaggacgcggccatcgtgggctacaaggactcgccctccgtctgggccgccgtccccgggaaaacgttcgtcaacatcacgccagctgaggtgggtgtcctggttggcaaagaccggtcaagtttttacgtgaatgggctgacacttgggggccagaaatgttcggtgatccgggactcactgctgcaggatggggaatttagcatggatcttcgtaccaagagcaccggtggggcccccaccttcaatgtcactgtcaccaagactgacaagacgctagtcctgctgatgggcaaagaaggtgtccacggtggtttgatcaacaagaaatgttatgaaatggcctcccaccttcggcgttcccagtactgacctcgtctgtcccttccccttcaccgctccccacagctttgcacccctttcctccccatacacacacaaaccattttattttttgggccattaccccataccccttattgctgccaaaaccacatgggctgggggccagggctggatggacagacacctccccctacccatatccctcccgtgtgtggttggaaaacttttgttttttggggttttttttttctgaataaaaaagattctactaacaagg

(19) United States
(12) Patent Application Publication
(10) Pub. No.: US 2017/0129930 A1
(43) Pub. Date: May 11, 2017

(54) COMPOSITIONS AND METHODS FOR MODULATING NEURONAL DEGENERATION

(71) Applicant: Board of Trustees of the University of Arkansas, Little Rock, AR (US)

(72) Inventor: Mahmoud Kiaei, Little Rock, AR (US)

(73) Assignee: Board of Trustees of the University of Arkansas, Little Rock, AR (US)

(21) Appl. No.: 15/320,148

(22) PCT Filed: Jun. 19, 2015
(86) PCT No.: PCT/US15/36755

§ 371 Date: Dec. 19, 2016

Related U.S. Application Data
(60) Provisional application No. 62/014,306, filed on Jun. 19, 2014.

(51) Int. Cl. C07K 14/47; A01K 67/027; A61K 49/00

(52) U.S. Cl. CPC C07K 14/4716; A61K 49/0008; A01K 67/0278; A01K 2207/15; A01K 2217/072; A01K 2227/105; A01K 2267/0318

(57) ABSTRACT

The present disclosure provides genetically modified animals and cells comprising a polynucleotide encoding human profilin1. Also provided are methods of assessing the effects of agents in genetically modified animals and cells comprising a polynucleotide encoding human profilin1.

### CROSS REFERENCE TO RELATED APPLICATIONS

This application claims the priority of U.S. provisional application No. 62/014,306, filed Jun. 19, 2014, which is hereby incorporated by reference in its entirety.

### GOVERNMENTAL RIGHTS

This invention was made with government support under P30 GM110702 and NS088653 awarded by the NIH. The government has certain rights in the invention.

### FIELD OF THE INVENTION

This invention generally relates to genetically modified animals or cells comprising a polynucleotide encoding a human profilin1 protein. In particular, the invention relates to a genetically modified animal comprising a polynucleotide encoding a human profilin1 protein, wherein the animal develops amyotrophic lateral sclerosis (ALS).

### BACKGROUND OF THE INVENTION

Amyotrophic lateral sclerosis (ALS) is a fatal disease resulting from progressive degeneration of motor neurons and affects 30,000 Americans each year. ALS was discovered over 140 years ago, and the mechanisms causing the neurodegeneration are still not fully understood. Animal models developed or being developed based on genes with mutations linked to ALS (e.g., SOD1, TARDBP, FUS/TLS, C9orf72) have advanced our understanding of the disease mechanisms but have not resulted in development of effective therapeutic interventions for ALS. Mutations in the gene for a protein called profilin1 are reported to be the cause of ALS in a subpopulation of familial ALS. Profilin1 is a protein that plays an important role cellular cytoskeleton and in nerve cells for the growth of long nerve fibers called axons which are adversely affected by ALS. Therefore, there is a need for a novel ALS animal model with utility for better understanding the disease and developing new therapeutic strategies to treat ALS.

### SUMMARY OF THE INVENTION

One aspect of the present disclosure encompasses a genetically modified animal comprising at least one exogenous nucleic acid, wherein the exogenous nucleic acid comprises a polynucleotide encoding a human profilin1 protein.

A further aspect provides a genetically modified animal cell. The cell comprises at least one exogenous nucleic acid, wherein the exogenous nucleic acid comprises a polynucleotide encoding a human profilin1 protein.

In another aspect, the invention provides a method for assessing the therapeutic potential of an agent on an animal. The method comprises administering an agent to a genetically modified animal comprising at least one exogenous nucleic acid, wherein the exogenous nucleic acid comprises a polynucleotide encoding a human profilin1 protein and comparing the results of a selected parameter to results obtained from a second genetically modified animal which was not administered the agent. The selected parameter is chosen from: weight loss, hindlimb muscle atrophy, histopathology, behavior and premature death.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts the predicted structure of human β-actin (green) and PFN1 (yellow, red, blue) complex, showing binding of actin to PFN1 and the critical position of glycine 118 near the actin-PFN1 binding site.

FIG. 2 depicts a gel showing high (H), medium (M) or low (L) expression of human PFN1 DNA in PFN1 Founder lines.

FIG. 3 depicts a Western blot revealing bands of human and mouse PFN1 protein in the spinal cord of mutant PFN1 transgenic founder lines (H, M, L). Wildtype (WT) control mice express only mouse PFN1. GAPDH served as loading control.

FIG. 4 depicts a graph showing body weights of PFN1 Founder H, F1 female and wildtype control mice. F1 mouse died at 179 days of age. Since n=1 from each strain, statistical analysis was not performed.

FIG. 5 depicts images showing hindlimb skeletal muscle atrophy in Founder H and F1 mice shown as compared to WT control.

FIG. 6 depicts histopathology of human PFN1 expression and astrocytosis (GFAP staining) in the CNS of Founder H and wildtype control mice.

FIG. 7A-B depicts images of the behavior of wildtype and PFN1 transgenic mice. (FIG. 7A) Snap shots of video from Founder H and wildtype control mice. Hindlimb clasping, decreased movement, and altered walking posture at 6 months is evident for Founder H compared to wildtype. (FIG. 7B) F1s from H line are included that show phenotypes. The F1 female 2 shown here died of ALS at 179 days after birth.

FIG. 8 depicts pictures from F1s from H line mice included that show phenotypes. The F1 female shown here is 179 days of age and expected to die in 7-10 days from this date.

FIG. 9 depicts inked footprints showing walking behavior and stride length. Founder H at 180 days of age has drastically large reduction (54 mm) stride length, while L2 experiencing 24 mm reduction. F1s' stride length prints show drastic reduction at 167 days after birth.

FIG. 10A-C depicts transgene DNA and protein expression in F1 hPFN1^{G118V} mice. (FIG. 10A) PCR of endogenous mouse and mutant human PFN1 genes. (FIG. 10B) Western blot of endogenous mouse and mutant human PFN1 proteins. (FIG. 10C) PFN1 expression in overexpressing wild type hPFN1 mice as control for mutant line. hPFN1 migrates slightly slower than mouse PFN1 so they run as a doublet. hPFN1=human profilin1 WT=Non-transgenic.

FIG. 11A-D depicts images and a graph showing motor neuron degeneration in mutant hPFN1^{G118V} mice. Stereological cell counts of motor neurons in the motor horn (indicated in the images by the circle) reveal that the cell number is reduced compared to wt (early stage 140 days and endstage 178 days of age). (FIG. 11A) Wild-type; (FIG. 11B) PFN1 Early stage; (FIG. 11C) PFN1 Endstage; (FIG. 11D) Graphical representation of the images. Data is normalized to the number of neurons in wt mice. Data was analyzed by ANOVA. n=6/group. Scale bar=100 μM.

FIG. 12A-D depicts images showing mutant PFN1^{G118V} mice with ventral motor axons degeneration and mitochondria membrane fragmentation. Wild-type mice demonstrate normal axons (FIG. 12A) and normal mitochondria (FIG. 12B). In the hPFN1^{G118V} mouse axons (asterisks in FIG. 12C) are distorted and show degenerative swelling and shrinkage, and mitochondria (FIG. 12D) demonstrate membrane blebbing and fragmentation as well as disorganized cristae (arrows). Animals were 165 days old. Scale bars=5 μm in panels FIG. 12A and FIG. 12C and 100 nm in FIG. 12B inset and FIG. 12D.

FIG. 13A-D depicts images showing mutant hPFN1^{G118V} mice gait is impaired. (FIG. 13A-B) Gait measurement with the CatWalk shows dramatic differences between hPFN1^{G118V} (FIG. 13B) and non-Tg mice (FIG. 13A) (see also Table 1). (FIG. 13C-D) Inked footprints on paper show shorter stride length and abnormal gait in hPFN1^{G118V} mice (FIG. 13D) relative to non-Tg mice (FIG. 13C). Animals were 175 days old when tested.

FIG. 14 depicts a graph showing progressive motor weakness and performance of hPFN1^{G118V} mice compared to Non-Tg control as determined by Rotarod. n=15/group.

FIG. 15 depicts a graph showing hPFN1^{G118V} mice exhibit premature death. n=15/group.

FIG. 16A-B depicts images and a graph showing mutant PFN1^{G118V} spinal cord show decrease in F-actin and increase in G-actin. (FIG. 16A) Phalloidin stain (green) for F-actin and DNase I stain (red) for G-actin in spinal cord ventral horns of PFN1 mice at 155 days of age as compared to controls. (FIG. 16B) Relative fluorescence F/G ratio is lower in hPFN1G118V. Arrow point to a motor neuron with high G-actin. Student's t-test, *P<0.05, n=4.

FIG. 17 depicts a graph showing mutant hPFN1^{G118V} form aggregates in the spinal cord. NP-40 Sol/Insoluble fractions from total homogenates shows hPFN1 band while mouse PFN1 is absent. Values are expressed relative to GAPDH control. N=2 per genotype, *P<0.05 relative to Non-Tg (I) and WT-Tg(I). S=Soluble, I=Insoluble

FIG. 18 depicts a graphical representation of the stride length of WT, H, L1 and L2 founders depicted in FIG. 9.

FIG. 19 depicts images showing mutant PFN1^{G118V} spinal cord show decrease in F-actin and increase in G-actin. Phalloidin stain (green) for F-actin and DNase I stain (red) for G-actin in spinal cord ventral horns of PFN1 mice at 155 days of age as compared to controls.

### DETAILED DESCRIPTION OF THE INVENTION

The present disclosure provides a genetically modified animal or animal cell comprising at least one exogenous nucleic acid, wherein the nucleic acid comprises a polynucleotide encoding a human profilin1 protein. A genetically modified animal comprising at least one exogenous nucleic acid may be termed a “knock in” or a “conditional knock in.” As detailed below, a knock in animal may be a humanized animal. Furthermore, a genetically modified animal comprising at least one exogenous nucleic acid, wherein the nucleic acid comprises a polynucleotide encoding a human profilin1 protein may comprise a targeted point mutation(s) or other modification such that an altered protein product is produced. Briefly, the process comprises introducing into an embryo or cell at least one nucleic acid comprising a polynucleotide encoding a human profilin1 protein. The method further comprises incubating the embryo or cell to allow expression of the human profilin1 protein, wherein the animal develops amyotrophic lateral sclerosis (ALS). The genetically modified animal may be useful in the development and screening of therapeutically useful reagents as well as studying the biological mechanisms underlying ALS caused by or linked to a mutation in human profilin1.

### I. Genetically Modified Animals

One aspect of the present disclosure provides a genetically modified animal comprising at least one exogenous nucleic acid, wherein the nucleic acid comprises a polynucleotide encoding a human profilin1 protein. Profilin1 is a cytoskeletal protein that binds actin monomers, regulating actin polymerization and cytoskeletal assembly. Human profilin1 is encoded by PFN1 nucleic acid. Human profilin1 comprises the amino acid sequence set forth in NM_005022.3 (SEQ ID NO:1 MAGWNAYIDNLMADGTCQDAAIVGYKDSPSVWAAVPGKTFVNITPAEVGVLVGKDRS SFYVNGLTLGGQKCSVIRDSLLQDGEFSMDLRTKSTGGAPTFNVTVTKTDKTLVLLMG KEGVHGGLINKKCYEMASHLRRSQY). PFN1 nucleic acid comprises the polynucleotide sequence set forth in NM_005022.3 (SEQ ID NO:2 cccgcagggt ccacacgggt cgggccgggc gcgctcccgt gcagccggct ccggccccga ccgccccatg cactcccggc cccggcgcag gcgcaggcgc gggcacacgc gccgccgccc gccggtcctt cccttcggcg gaggtggggg aaggaggagt catcccgttt aaccctgggc tccccgaact ctccttaatt tgctaaattt gcagcttgct aattcctcct gctttctcct tccttccttc ttctggctca ctccctgccc cgataccaaa gtctggttta tattcagtgc aaattggagc aaaccctacc cttcacctct ctcccgccac cccccatcct tctgcattgc tttccatcga actctgcaaa ttttgcaata gggggaggga tttttaaaat tgcatttgca aagttcggtg tctgggctgg cgagtggggg agggagggaa tggggagtag gccccgcccc taccgtcctt tgcaaataaa aatctagcgg ggcggggggg gggaggagca ggaagtggcg gtgcgagggc tgctgcacag cgagcggagc cgcggtccgg acggcagcgc gtgccccgag ctctccgcct ccccccgccc gccagccgag gcagctcgag cccagtccgc ggccccagca gcagcgccga gagcagcccc agtagcagcg ccatggccgg gtggaacgcc tacatcgaca acctcatggc ggacgggacc tgtcaggacg cggccatcgt gggctacaag gactcgccct ccgtctgggc cgccgtcccc gggaaaacgt tcgtcaacat cacgccagct gaggtgggtg tcctggttgg caaagaccgg tcaagttttt acgtgaatgg gctgacactt gggggccaga aatgttcggt gatccgggac tcactgctgc aggatgggga atttagcatg gatcttcgta ccaagagcac cggtggggcc cccaccttca atgtcactgt caccaagact gacaagacgc tagtcctgct gatgggcaaa gaaggtgtcc acggtggttt gatcaacaag aaatgttatg aaatggcctc ccaccttcgg cgttcccagt actgacctcg tctgtccctt ccccttcacc gctccccaca gctttgcacc cctttcctcc ccatacacac acaaaccatt ttattttttg ggccattacc ccatacccct tattgctgcc aaaaccacat gggctggggg ccagggctgg atggacagac acctccccct acccatatcc ctcccgtgtg tggttggaaa acttttgttt tttggggttt tttttttctg aataaaaaag attctactaa caagg).

In some embodiments, Human profilin1 consists of the amino acid sequence set forth in NM_005022.3 (SEQ ID NO:1 MAGWNAYIDNLMADGTCQDAAIVGYKDSPSVWAAVPGKTFVNITPAEVGVLVGKDRS SFYVNGLTLGGQKCSVIRDSLLQDGEFSMDLRTKSTGGAPTFNVTVTKTDKTLVLLMG KEGVHGGLINKKCYEMASHLRRSQY). Similarly, in certain embodiments, PFN1 nucleic acid consists of the polynucleotide sequence set forth in NM_005022.3 (SEQ ID NO:2 cccgcagggt ccacacgggt cgggccgggc gcgctcccgt gcagccggct ccggccccga ccgccccatg cactcccggc cccggcgcag gcgcaggcgc gggcacacgc gccgccgccc gccggtcctt cccttcggcg gaggtggggg aaggaggagt catcccgttt aaccctgggc tccccgaact ctccttaatt tgctaaattt gcagcttgct aattcctcct gctttctcct tccttccttc ttctggctca ctccctgccc cgataccaaa gtctggttta tattcagtgc aaattggagc aaaccctacc cttcacctct ctcccgccac cccccatcct tctgcattgc tttccatcga actctgcaaa ttttgcaata gggggaggga tttttaaaat tgcatttgca aagttcggtg tctgggctgg cgagtggggg agggagggaa tggggagtag gccccgcccc taccgtcctt tgcaaataaa aatctagcgg ggcggggggg gggaggagca ggaagtggcg gtgcgagggc tgctgcacag cgagcggagc cgcggtccgg acggcagcgc gtgccccgag ctctccgcct ccccccgccc gccagccgag gcagctcgag cccagtccgc ggccccagca gcagcgccga gagcagcccc agtagcagcg ccatggccgg gtggaacgcc tacatcgaca acctcatggc ggacgggacc tgtcaggacg cggccatcgt gggctacaag gactcgccct ccgtctgggc cgccgtcccc gggaaaacgt tcgtcaacat cacgccagct gaggtgggtg tcctggttgg caaagaccgg tcaagttttt acgtgaatgg gctgacactt gggggccaga aatgttcggt gatccgggac tcactgctgc aggatgggga atttagcatg gatcttcgta ccaagagcac cggtggggcc cccaccttca atgtcactgt caccaagact gacaagacgc tagtcctgct gatgggcaaa gaaggtgtcc acggtggttt gatcaacaag aaatgttatg aaatggcctc ccaccttcgg cgttcccagt actgacctcg tctgtccctt ccccttcacc gctccccaca gctttgcacc cctttcctcc ccatacacac acaaaccatt ttattttttg ggccattacc ccatacccct tattgctgcc aaaaccacat gggctggggg ccagggctgg atggacagac acctccccct acccatatcc ctcccgtgtg tggttggaaa acttttgttt tttggggttt tttttttctg aataaaaaag attctactaa caagg).

In still further embodiments, Human profilin1 may refer to a homologous sequence to either SEQ ID NO:1 or SEQ ID NO:2. For instance, Human profilin1 may be at least 80, 85, 90, or 95% homologous to SEQ ID NO:1 or SEQ ID NO:2. In one embodiment, Human profilin1 may be at least 80, 81, 82, 83, 84, 85, 86, 87, 88, or 89% homologous to SEQ ID NO:1 or SEQ ID NO:2. In another embodiment, Human profilin1 of the invention may be at least 90, 91, 92, 93, 94, 95, 96, 97, 98, 99, or 100% homologous to SEQ ID NO:1 or SEQ ID NO:2.

In determining whether Human profilin1 has significant homology or shares a certain percentage of sequence identity with SEQ ID NO:1 or SEQ ID NO:2, sequence similarity may be determined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the invention. Equally, BLAST protein searches may be performed with the BLASTX program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed. See www.ncbi.nlm.nih.gov for more details.

As used herein, the terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

In an embodiment, the polynucleotide may encode for the wild-type form of human profilin1 protein. In another embodiment, the polynucleotide may be modified such that it encodes for a mutated form of human profilin1 protein. For example, the polynucleotide encoding for a human profilin1 protein may comprise at least one modification such that a mutated version of the protein is produced. As such, the polynucleotide may be modified such that at least one nucleotide is changed and the expressed human profilin1 protein comprises at least one changed amino acid residue. The polynucleotide may be modified to comprise more than one nucleotide change such that more than one amino acid is changed. For example, the polynucleotide may comprise two, three, four, or more specific nucleotide changes such that the encoded protein comprises one, two, three, four, or more amino acid changes. Additionally, the polynucleotide may be modified to have a three nucleotide deletion or insertion such that the expressed human profilin1 protein comprises a single amino acid deletion or insertion, provided such a protein is functional. The modified protein may have altered substrate specificity, altered enzyme activity, altered kinetic rates, and so forth. In a preferred embodiment, the polynucleotide may be modified such that it encodes for a mutated human profilin1 protein comprising a mutation selected from the group consisting of C71G, E117G and G118V relative to SEQ ID NO:1. Stated another way, a mutation at amino acid position 71 replaces cysteine (C, cys) with glycine (G, gly); a mutation at amino acid position 117 replaces glutamic acid (E, glu) with glycine (G, gly); and a mutation at position 118 replaces glycine (G, gly) with valine (V, val) relative to SEQ ID NO:1. In an exemplary embodiment, the polynucleotide may be modified such that it encodes for a mutated human profilin1 protein comprising G118V relative to SEQ ID NO:1. One of skill in the art would be able to construct a polynucleotide as described herein using well-known standard recombinant techniques (see, for example, Sambrook et al., 2001 and Ausubel et al., 1996).

In another embodiment, the exogenous nucleic acid may be operably linked to a regulatory sequence. A suitable regulatory sequence may be a promoter. The term “promoter”, as used herein, may mean a synthetic or naturally-derived molecule that is capable of conferring, activating or enhancing expression of a nucleic acid. A promoter is a critical sequence required for regulating the spatial and temporal expression pattern of an exogenous nucleic acid. Promoter sequences are isolated from upstream regions of endogenous mammalian genes. A promoter sequence normally includes a transcriptional start site as well as transcription regulatory sequences. Many promoters have been reported that can successfully achieve tissue-specific and developmental stage-specific expression of nucleic acids. A database search such as PubMed or International Mouse Strain Resource is a preferable way to find out the promoters of tissue or cell type of interest. Promoters should be tested to determine if they contain the appropriate transcriptional regulatory elements. A large number of promoters have been characterized that direct a wide variety of nucleic acid expression patterns. The best way to choose a promoter to generate genetically modified animals is to review original papers that examined endogenous expression patterns. A promoter may be constitutive, inducible/repressible or cell type specific. In certain embodiments, the promoter may be constitutive. Non-limiting examples of constitutive promoters include CMV, UBC, EF1a, SV40, PGK, CAG, CBA/CAGGS/ACTB, CBh, MeCP2, U6 and H1. In other embodiments, the promoter may be repressible such as the tetracycline-controlled system. In another embodiment, the promoter may be inducible such as the doxycycline-inducible system. In certain embodiments, the promoter may be cell type specific. Non-limiting cell type specific promoters syapsin, SYN1, NSE/RU5′ (neurons), chromogranin A (neuroendocrine cells), desmin, Mb (muscle cells), GFAP (astrocytes). In an embodiment, the exogenous nucleic acid is operably linked to the human profilin1 promoter. In an exemplary embodiment, the exogenous nucleic acid is operably linked to a mouse prion promoter.

The term “operably linked,” as used herein, means that expression of a nucleic acid sequence is under the control of a promoter with which it is spatially connected. A promoter may be positioned 5′ (upstream) of the nucleic acid sequence under its control. The distance between the promoter and a nucleic acid sequence to be expressed may be approximately the same as the distance between that promoter and the native nucleic acid sequence it controls. As is known in the art, variation in this distance may be accommodated without loss of promoter function.

A promoter of the invention should achieve expression in the central nervous system (CNS). According to the invention, the human profilin1 protein may be expressed in the central nervous system (CNS) comprising the brain and spinal cord. Additionally, the human profilin1 protein may be expressed in skeletal muscle. In an exemplary embodiment, the human profilin1 protein is expressed in the brain, spinal cord and skeletal muscle.

In a preferred embodiment, the exogenous nucleic acid is overexpressed relative to the endogenous nucleic acid encoding for the endogenous profilin1 protein. For example, the exogenous nucleic acid may be overexpressed by from about 20 fold to about 1.5 fold relative to the endogenous nucleic acid encoding for the endogenous profilin1 protein. In another embodiment, the exogenous nucleic acid may be overexpressed by from about 10 fold to about 1.5 fold relative to the endogenous nucleic acid encoding for the endogenous profilin1 protein. In still another embodiment, the exogenous nucleic acid may be overexpressed by from about 5 fold to about 1.5 fold relative to the endogenous nucleic acid encoding for the endogenous profilin1 protein. As such, the exogenous nucleic acid may be overexpressed by about 20, about 19, about 18, about 17, about 16, about 15, about 14, about 13, about 12 or about 11 fold relative to the endogenous nucleic acid encoding for the endogenous profilin1 protein. Additionally, the exogenous nucleic acid may be overexpressed by about 10, about 9, about 8, about 7, about 6, about 5, about 4, about 3, about 2 or about 1.5 fold relative to the endogenous nucleic acid encoding for the endogenous profilin1 protein. Overexpression of nucleic acid may be determined by methods known in the art. For example, nucleic acid may be run on an agarose gel, stained with ethidium bromide, photographed and analyzed for densitometry of the bands. In another preferred embodiment, the human profilin1 protein is overexpressed relative to the endogenous profilin1 protein. For example, the human profilin1 protein may be overexpressed by from about 20 fold to about 1.5 fold relative to the endogenous profilin1 protein. In another embodiment, the human profilin1 protein may be overexpressed by from about 10 fold to about 1.5 fold relative to the endogenous profilin1 protein. As such, the human profilin1 protein may be overexpressed by about 20, about 19, about 18, about 17, about 16, about 15, about 14, about 13, about 12 or about 11 fold relative to the endogenous profilin1 protein. Additionally, the human profilin1 protein may be overexpressed by about 10, about 9, about 8, about 7, about 6, about 5, about 4, about 3, about 2 or about 1.5 fold relative to the endogenous profilin1 protein. Overexpression of protein may be determined by methods known in the art. For example, a Western blot may be performed to identify protein levels.

According to the invention, a genetically modified animal of the invention may develop a neurodegenerative disease. A neurodegenerative disease may include, but not limited to, ALS, Parkinson's disease (PD) and PD-related disorders, Alzheimer's disease (AD) and other dementias, Prion disease, Creutzfeld-Jakob disease, Motor neuron diseases (MND), Huntington's disease (HD), spinocerebellar ataxia (SCA), spinal muscular atrophy (SMA), Friedrich's ataxia, Lewy body disease, and multiple sclerosis (MS). A neurodegenerative disease of the invention may be characterized by axonal degeneration, mitochondrial abnormalities and/or muscle weakness and degeneration. In an exemplary embodiment, a genetically modified animal of the invention develops amyotrophic lateral sclerosis (ALS). Development of ALS may be characterized by animal weight loss, hindlimb muscle atrophy, histopathology, behavior, and premature death. Histopathology may be used to examine human profilin1 protein expression. More specifically, human profilin1 protein expression may be examined in the central nervous system (CNS) of the animal, such as the brain and spinal cord. Additionally, histopathology may be used to examine the presence of astrocytosis. Astrocytosis is an increase in the number of neuroglial cells with fibrous or protoplasmic processes frequently observed in an irregular area adjacent to degenerative lesions, such as abscesses, certain brain neoplasms, and encephalomalacia. Astrocytosis represents a reparative process and in some cases may be diffuse in a large region. Staining with an astrocyte marker, such as glial fibrillary acidic protein (GFAP), may reveal astrocytosis. Behavior may also be examined as an indication of the development of ALS. ALS-like behavioral phenotypes may include, but are not limited to, hindlimb tremor, progressive clasping, hindlimb muscle weakness, reduced stride length, lower gait, hypokinetic behavior, reduced motor performance including inability to stay on a rotating rod, and abnormal walking posture such as a hunched back. Other methods to diagnose the development of ALS are known in the art and may include electrodiagnostic tests including electomyography (EMG) and nerve conduction velocity (NCV), blood and urine studies including high resolution serum protein electrophoresis, thyroid and parathyroid hormone levels and 24-hour urine collection for heavy metals, spinal tap, x-rays, including magnetic resonance imaging (MRI), myelogram of cervical spine, muscle and/or nerve biopsy, and thorough neurological examination.

Additionally, the polynucleotide encoding a human profilin1 protein may be modified to include a tag or reporter gene as are well-known. Reporter genes include those encoding selectable markers such as chloramphenicol acetyltransferase (CAT) and neomycin phosphotransferase (neo), and those encoding a fluorescent protein, luciferase, alkaline phosphatase, beta-galactosidase, beta-lactamase, horseradish peroxidase, and variants thereof. A fluorescent protein may include green fluorescent protein (GFP), red fluorescent protein, or any genetically engineered variant thereof that improves the reporter performance. Non-limiting examples of known such FP variants include EGFP, blue fluorescent protein (EBFP, EBFP2, Azurite, mKalama1), cyan fluorescent protein (ECFP, Cerulean, CyPet) and yellow fluorescent protein derivatives (YFP, Citrine, Venus, YPet). For example, in a genetic construct containing a reporter gene, the reporter gene sequence can be fused directly to the polynucleotide encoding a human profilin1 protein to create a fusion. A reporter sequence can be integrated in a targeted manner in the polynucleotide encoding a human profilin1 protein, for example the reporter sequence may be integrated specifically at the 5′ or 3′ end of the polynucleotide encoding a human profilin1 protein. The two sequences are thus under the control of the same promoter elements and are transcribed into a single messenger RNA molecule.

In an embodiment, the at least one exogenous nucleic acid, wherein the nucleic acid comprises a polynucleotide encoding a human profilin1 protein may be chromosomally integrated into the genetically modified animal. For example, a polynucleotide encoding a human profilin1 protein may be integrated into a chromosomal sequence encoding a protein such that the chromosomal sequence is inactivated, but wherein the exogenous polynucleotide may be expressed. In such a case, the polynucleotide encoding the human profilin1 protein may be operably linked to a promoter control sequence as described above. Alternatively, a polynucleotide encoding a human profilin1 protein may be integrated into a chromosomal sequence without affecting expression of a chromosomal sequence. For example, a polynucleotide encoding a human profilin1 protein may be integrated into a “safe harbor” locus, such as the Rosa26 locus, HPRT locus, or AAV locus. An animal comprising a chromosomally integrated polynucleotide encoding a human profilin1 protein may be called a “knock-in,” and it should be understood that in certain iterations of the disclosure such an animal may have no selectable marker. The knock-in animal may be heterozygous for chromosomally integrated polynucleotide encoding a human profilin1 protein. Alternatively, the knock-in animal may be homozygous for the chromosomally integrated polynucleotide encoding a human profilin1 protein.

Optionally, the genetically modified animal may be a “humanized” animal comprising at least one exogenous nucleic acid, wherein the nucleic acid comprises a polynucleotide encoding a human profilin1 protein. The functional profilin1 protein may have no corresponding ortholog in the genetically modified animal. Alternatively, the wild-type animal from which the genetically modified animal is derived may comprise an ortholog corresponding to the functional profilin1 protein. In this case, the orthologous sequence in the “humanized” animal is inactivated such that no functional protein is made and the “humanized” animal comprises at least one exogenous nucleic acid, wherein the nucleic acid comprises a polynucleotide encoding a human profilin1 protein. Those of skill in the art appreciate that “humanized” animals may be generated by crossing a knock out animal with a knock in animal comprising the at least one exogenous nucleic acid, wherein the nucleic acid comprises a polynucleotide encoding a human profilin1 protein.

A “knock out” or a “conditional knock out” animal is a genetically modified animal comprising at least one exogenous nucleic acid, wherein the nucleic acid comprises a polynucleotide encoding for an inactivated profilin1 protein that is endogenous to that animal. The polynucleotide encoding for an inactivated profilin1 protein may include a deletion mutation (i.e., deletion of one or more nucleotides), an insertion mutation (i.e., insertion of one or more nucleotides), or a nonsense mutation (i.e., substitution of a single nucleotide for another nucleotide such that a stop codon is introduced). As a consequence of the mutation, the targeted profilin1 is inactivated and a functional profilin1 protein is not produced. The knock out animal comprises no endogenously expressed profilin1.

In yet another embodiment, the expression pattern of the human profilin1 protein may be altered using a conditional knockout system. A non-limiting example of a conditional knockout system includes a Cre-lox recombination system. A Cre-lox recombination system comprises a Cre recombinase enzyme, a site-specific DNA recombinase that can catalyze the recombination of a nucleic acid sequence between specific sites (lox sites) in a nucleic acid molecule. Methods of using this system to produce temporal and tissue specific expression are known in the art. In general, a genetically modified animal is generated with lox sites flanking a chromosomally integrated polynucleotide, such as a chromosomally integrated polynucleotide encoding a human profilin1 protein. The genetically modified animal comprising the lox-flanked chromosomally integrated polynucleotide encoding a human profilin1 protein may then be crossed with another genetically modified animal expressing Cre recombinase. Progeny animals comprising the lox-flanked chromosomally integrated polynucleotide and the Cre recombinase are then produced, and the lox-flanked chromosomally integrated polynucleotide encoding a human profilin1 protein is recombined, leading to deletion or inversion of the chromosomally integrated polynucleotide encoding the protein. Expression of Cre recombinase may be temporally and conditionally regulated to effect temporally and conditionally regulated recombination of the chromosomally integrated polynucleotide encoding a human profilin1 protein.

### (a) Animals

The term “animal,” as used herein, refers to a non-human animal. The animal may be an embryo, a juvenile, or an adult. Suitable animals include vertebrates such as mammals, birds, reptiles, amphibians, and fish. Examples of suitable mammals include without limit rodents, companion animals, livestock, and primates. Non-limiting examples of rodents include mice, rats, hamsters, gerbils, and guinea pigs. Suitable companion animals include but are not limited to cats, dogs, rabbits, hedgehogs, and ferrets. Non-limiting examples of livestock include horses, goats, sheep, swine, cattle, llamas, and alpacas. Suitable primates include but are not limited to capuchin monkeys, chimpanzees, lemurs, macaques, marmosets, tamarins, spider monkeys, squirrel monkeys, and vervet monkeys. Non-limiting examples of birds include chickens, turkeys, ducks, and geese. An exemplary animal is a mouse. Non-limiting examples of suitable mouse strains include 129, A/J, AKR, BALB/c, C3H/HeJ, C3H/HeN, C57BL/6, C57BL/10, CBA, DBA, FVB, ICR, NOD, and SJL.

### II. Genetically Modified Cells

A further aspect of the present disclosure provides genetically modified cells or cell lines comprising at least one exogenous nucleic acid, wherein the exogenous nucleic acid comprises a polynucleotide encoding a human profilin1 protein. The genetically modified cell or cell line may be derived from any of the genetically modified animals disclosed herein. For example, the genetically modified cell may be a sperm cell or an ES cell derived from a genetically modified animal disclosed herein. Additionally, the genetically modified cells may be an embryo or ovaries from a genetically modified animal. Alternatively, the exogenous nucleic acid comprising a polynucleotide encoding a human profilin1 protein may be edited in a cell as detailed below. The disclosure also encompasses a lysate of said cells or cell lines.

In general, the cells will be eukaryotic cells. Suitable host cells include fungi or yeast, such as Pichia, Saccharomyces, or Schizosaccharomyces; insect cells, such as SF9 cells from Spodoptera frugiperda or S2 cells from Drosophila melanogaster; and animal cells, such as mouse, rat, hamster, non-human primate, or human cells. Exemplary cells are mammalian. The mammalian cells may be primary cells. The cells may be of a variety of cell types, e.g., fibroblast, myoblast, T or B cell, macrophage, epithelial cell, and so forth.

When mammalian cell lines are used, the cell line may be any established cell line or a primary cell line that is not yet described. The cell line may be adherent or non-adherent, or the cell line may be grown under conditions that encourage adherent, non-adherent or organotypic growth using standard techniques known to individuals skilled in the art. Non-limiting examples of suitable mammalian cell lines include Chinese hamster ovary (CHO) cells, monkey kidney CVI line transformed by SV40 (COS7), human embryonic kidney line 293, baby hamster kidney cells (BHK), mouse sertoli cells (TM4), monkey kidney cells (CV1-76), African green monkey kidney cells (VERO), human cervical carcinoma cells (HeLa), canine kidney cells (MDCK), buffalo rat liver cells (BRL 3A), human lung cells (W138), human liver cells (Hep G2), mouse mammary tumor cells (MMT), rat hepatoma cells (HTC), HIH/3T3 cells, the human U2-OS osteosarcoma cell line, the human A549 cell line, the human K562 cell line, the human HEK293 cell lines, the human HEK293T cell line, and TR1 cells. For an extensive list of mammalian cell lines, those of ordinary skill in the art may refer to the American Type Culture Collection catalog (ATCC®, Manassas, Va.).

In still other embodiments, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, and unipotent stem cells.

### III. Generating a Genetically Modified Animal or Cell

In general, the genetically modified animal or cell detailed above in Section I and Section II, respectively, is generated by methods conventional in the art, particularly in animals such as mice or rats, as described, for example, in U.S. Pat. Nos. 4,736,866 and 4,870,009. Typically, the process for generating a genetically modified animal or cell comprises: (a) introducing into an embryo or cell at least one exogenous nucleic acid, wherein the exogenous nucleic acid comprises a polynucleotide encoding a human profilin1 protein; and (b) culturing the embryo or cell to allow expression of the human profilin1 protein.

Typically, the exogenous nucleic acid will be DNA. The exogenous nucleic acid may be a DNA plasmid, a bacterial artificial chromosome (BAC), a yeast artificial chromosome (YAC), a viral vector, a linear piece of DNA, a PCR fragment, a naked nucleic acid, or a nucleic acid complexed with a delivery vehicle such as a liposome or poloxamer. An exemplary exogenous nucleic acid comprising a polynucleotide encoding a human profilin1 protein may be a plasmid.

To generate a genetically modified animal or cell, at least one exogenous nucleic acid, wherein the exogenous nucleic acid comprises a polynucleotide encoding a human profilin1 protein is delivered to the embryo or the cell of interest. Typically, the embryo is a fertilized one-cell stage embryo of the species of interest. Suitable methods of introducing the exogenous nucleic acid to the embryo or cell include microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In one embodiment, the exogenous nucleic acid may be introduced into an embryo by microinjection. The exogenous nucleic acid may be microinjected into the nucleus or the cytoplasm of the embryo.

The method of generating a genetically modified animal or cell further comprises culturing the embryo or cell comprising the introduced nucleic acid(s) to allow expression of the human profilin1 protein. An embryo may be cultured in vitro (e.g., in cell culture). Typically, the embryo is cultured at an appropriate temperature and in appropriate media with the necessary O₂/CO₂ ratio to allow the expression of the zinc finger nuclease. Suitable non-limiting examples of media include M2, M16, KSOM, BMOC, and HTF media. A skilled artisan will appreciate that culture conditions can and will vary depending on the species of embryo. Routine optimization may be used, in all cases, to determine the best culture conditions for a particular species of embryo. In some cases, a cell line may be derived from an in vitro-cultured embryo (e.g., an embryonic stem cell line).

Alternatively, an embryo may be cultured in vivo by transferring the embryo into the uterus of a female host. Generally speaking the female host is from the same or similar species as the embryo. Preferably, the female host is pseudo-pregnant. Methods of preparing pseudo-pregnant female hosts are known in the art. Additionally, methods of transferring an embryo into a female host are known. Culturing an embryo in vivo permits the embryo to develop and may result in a live birth of an animal derived from the embryo. Such an animal would comprise a polynucleotide encoding the human profilin1 protein in every cell of the body.

Similarly, cells comprising the introduced nucleic acid(s) may be cultured using standard procedures to allow expression of the human profilin1 protein. Standard cell culture techniques are described, for example, in Santiago et al. (2008) PNAS105:5809-5814; Moehle et al. (2007) PNAS104:3055-3060; Urnov et al. (2005) Nature 435:646-651; and Lombardo et al (2007) Nat. Biotechnology 25:1298-1306. Those of skill in the art appreciate that methods for culturing cells are known in the art and can and will vary depending on the cell type. Routine optimization may be used, in all cases, to determine the best techniques for a particular cell type.

### IV. Applications

A further aspect of the present disclosure encompasses a method for assessing the therapeutic potential of an agent on an animal. Suitable agents include without limit pharmaceutically active ingredients, drugs, food additives, toxins, industrial chemicals, household chemicals, and other environmental chemicals. For example, the therapeutic potential of an agent may be measured in a genetically modified animal expressing mutant human profilin1 protein, such that the information gained therefrom may be used to predict the therapeutic potential of the agent in a human with a neurodegenerative disease. Neurodegenerative diseases are described in Section I. Specifically, the neurodegenerative disease may be ALS. In general, the method comprises administering an agent to a genetically modified animal comprising at least one exogenous nucleic acid, wherein the exogenous nucleic acid comprises a polynucleotide encoding a human profilin1 protein; and comparing results of a selected parameter to results obtained from a second genetically modified animal which was not administered the agent. Selected parameters include but are not limited to weight loss, hindlimb muscle atrophy, histopathology, behavior and premature death.

Methods of measuring weight loss, hindlimb muscle atrophy, histopathology, behavior and premature death are known in the art and are described in Section I. Additional parameters may include mitochondrial damage, oxidative stress and neuroinflammation. As such, genetically modified animals may be examined for mitochondrial structure and function, neuron loss, axonal damage, production of oxidative stress, glial activation, actin polymerization, proteasome dysfunction, excessive ubiquitination, and aggregation of SOD1, Ubiquilin2 and TDP-43.

A method for assessing the therapeutic potential of an agent on an animal may further comprise, determining if the agent abates the selected parameter. As used herein, the agent may “abate” the selected parameter if it lessens or decreases one or more parameter. For example, a genetically modified animal administered an agent may experience decreased weight loss, decreased hindlimb muscle atrophy, decreased histopathology associated with a neurodegenerative disease, decreased behavior modifications associated with a neurodegenerative disease and live longer compared to a second genetically modified animal not administered the agent. In a specific embodiment, the neurodegenerative disease may be ALS. The decrease may be significantly different compared to a genetically modified animal which was not administered the agent. A significant difference is enough of a difference to distinguish among the genetically modified animal administered the agent and the genetically modified animal not administered the agent, such as about 0.1%, 1%, 3%, 5%, 10%, 15%, 20%, 25%, 30%, or 40% or more difference between the genetically modified animal administered the agent and the genetically modified animal not administered the agent. In another embodiment the significant difference is measured using p-value. For instance, when using p-value, a parameter is identified as being significantly different between a genetically modified animal administered the agent and the genetically modified animal not administered the agent when the p-value is less than 0.1, preferably less than 0.05, more preferably less than 0.01, even more preferably less than 0.005, the most preferably less than 0.001.

Also provided are methods to assess the effect(s) of an agent in an isolated cell comprising at least polynucleotide encoding a human profilin1 protein, as well as methods of using lysates of such cells (or cells derived from a genetically modified animal disclosed herein) to assess the effect(s) of an agent.

In an aspect, the present disclosure provides a model of neurodegenerative disease comprising a genetically modified animal described herein. A neurodegenerative disease may include, but not limited to, ALS, Parkinson's disease (PD) and PD-related disorders, Alzheimer's disease (AD) and other dementias, Prion disease, Creutzfeld-Jakob disease, Motor neuron diseases (MND), Huntington's disease (HD), spinocerebellar ataxia (SCA), spinal muscular atrophy (SMA), Friedrich's ataxia, Lewy body disease, and multiple sclerosis (MS). In an embodiment, a genetically modified animal described herein may be used as a model to study axonal degeneration. In another embodiment, a genetically modified animal described herein may be used as a model to study mitochondrial abnormalities. In still another embodiment, a genetically modified animal described herein may be used as a model to study muscle weakness and degeneration. In an exemplary embodiment, a genetically modified animal described herein may be used as a model to study ALS.

Genetically modified animals may also be useful in methods for screening or evaluating new candidate therapeutic compounds or approaches, such as in screening of candidate therapeutic compounds for treating a neurodegenerative disease. In an embodiment, genetically modified animals described herein may be used for screening or evaluating new candidate therapeutic compounds or approaches for treating ALS. Genetically modified animals, such as for example knockout mice can be subjects for pre-clinical evaluation of a specific “gene therapy”. For example, genes may be introduced into hematopoietic progenitor cells, preferably into pluripotent stem cells with self-renewal capacity from patients with inherited genetic defects, or into pluripotent stem cells with self-renewal capacity from mouse models of patients with inherited genetic defects, and the cells re-introduced into the genetically modified mice for the purpose of determining therapeutic usefulness of the modified cells. Genetically modified animals may also be useful for studying the biological mechanisms underlying neuron degeneration. In an embodiment, genetically modified animals may also be useful for studying the biological mechanisms underlying ALS caused by or linked to a mutation in human profilin1.

### Examples

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Introduction for the Examples.

Amyotrophic lateral sclerosis (ALS) is a fatal disease resulting from progressive degeneration of motor neurons and affects 30,000 Americans each year. ALS was discovered over 140 years ago, and yet the mechanisms causing the neurodegeneration are not fully understood. However, development of transgenic mouse models carrying mutations in SOD1 or TDP-43 genes expanded our knowledge of the human disease. Non-rodent models also contributed greatly. We now know that multiple cellular pathologies (e.g., aggregation of mutant SOD1, TDP43, FUS, Ubiqulin2), mitochondrial dysfunction, neuroinflammation, and oxidative damage are major pathways that lead to the progressive death of motor neurons in the brain and spinal cord, resulting in paralysis of voluntary muscles. Despite this information, available preclinical and clinical models have failed to result in interventions that inhibit the toxic processes and motor neuron death in ALS. Further studies in developing viable animal models, which would enable us to find effective therapies for ALS, are greatly needed.

In a breakthrough study on familial ALS (fALS), a set of genetic mutations within the profilin 1 (PFN1) gene were found to be associated with the disease (Wu et al., 2012). At least four mutations were determined in the PFN1 gene in 5 families with 25 ALS patients, thus linking PFN1 to familial ALS. There is no data to show whether any sporadic ALS patients carry PFN1 mutations. PFN1 is a ubiquitously expressed actin-monomer binding protein involved in many cellular activities and regulates the formation of filamentous actin (F-actin) from monomeric actin (G-actin) molecules. F-actin is an essential component of axon structure (Witke et al., 2001), and PFN1 regulates G-actin polymerization into F-actin which is crucial for axon outgrowth. In addition, PFN1's interaction with phosphatidylinositols, such as phosphatidylinositolphosphates, may alter axonal trafficking in motor neurons, which is severely impaired in the later stages of ALS. PFN1 is also involved in transport of mitochondria, which interacts with microtubules and actin filaments via the myosin V motor linked factor (Hollenbeck and Saxton, 2005, Saxton and Hollenbeck, 2012).

There are four PFN isoforms in mammals (PFN1-4). PFN1 is suggested to be the prominent isoform expressed in the CNS. PFN1 is broadly expressed throughout all embryonic stages in rodents and is present in nearly all adult cell types and tissues (Witke et al., 1998). Major and conserved functions of PFN1 include the catalysis of nucleotide exchange of monomeric actin (G-actin) and the transfer of ATP-actin to the barbed end of actin filaments (F-actin) (Mockrin and Korn, 1980, Tilney et al., 1983). Profilin1 also plays an important role in mitochondrial transport, facilitating mitochondrial anchoring and movement. Profilin1 interacts with a large number of proteins including huntingtin (whose mutant gene is linked to Huntington's Disease), where it also inhibits polyglutamine aggregation (Goehler et al., 2004, Shao et al., 2008).

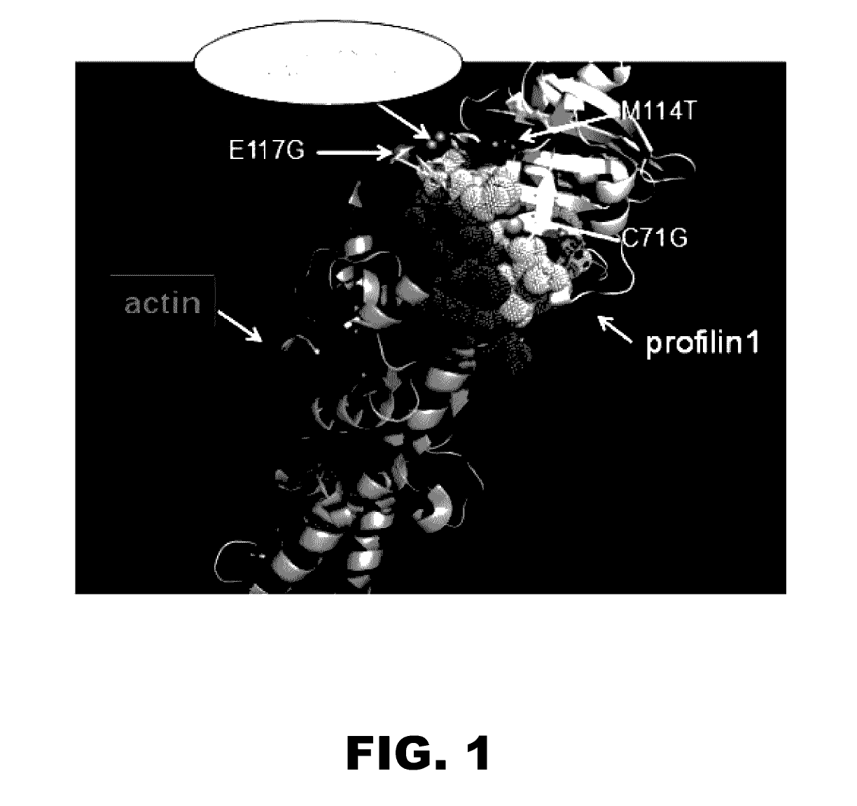

These PFN1 functions are relevant to the PFN1 mutations linked to ALS because axonal pathologies and mitochondrial abnormalities described in ALS may be due to changes in PFN1 function either by oxidative modification or mutation. Evidence is building for a critical role of PFN1 in motor neurons due to the fact that its mutation is now linked to ALS. Last year a landmark paper reported 4 mutations in the PFN1 gene in 276 subjects with the familial form of ALS (fALS) (Wu et al., 2012). Here we show the arrangement of profilin1-actin, including the binding surface of PFN1 with actin and the location of the 4 mutations, namely C71G; M114T; E117G; G118V, using a computer generated model (FIG. 1). We illustrate the critical locations of these mutations, which infers effects of the mutations on actin binding.

Collectively, these studies suggest that the PFN1 mutation is specific to a subset of familial ALS patients, similar to the SOD1 and TDP-43 mutants. Due to the known function of PFN1, investigating mutant profilin1 toxicity may shed light on ALS.

Several additional observations by Landers and colleagues link cytoskeletal pathway alterations to ALS pathogenesis. These investigators found that primary motor neurons expressing PFN1 mutations contained ubiquitinated, insoluble aggregates including the ALS-associated protein TDP-43. PFN1 mutants also display decreased PFN1-bound actin levels, which inhibits axonal outgrowth. Furthermore, primary motor neurons expressing mutant PFN1 display smaller growth cones with a reduced F/G-actin ratio (Wu et al., 2012). The potential link between ALS pathogenesis and the cytoskeletal system is poorly understood. In addition, PFN1's interaction with phosphatidylinositols like PIPs may alter axonal trafficking in motor neurons, which is severely impaired in the later stages of ALS. Profilin1 is also involved in transporting mitochondria since mitochondria interact with microtubules and actin filaments via the myosin V motor linked factor (Hallenbeck and Saxton, 2005, Saxton and Hollenbeck, 2012). It would be of great interest to examine the role of PFN1 mutations in the development of cytoskeletal system and axonal growth in order to better understand the disease mechanisms. Further studies involving this new target will shed light onto the mutant PFN1's role in axonal and neurite pathology, cytoskeletal structure, and mitochondrial dysfunction and transport associated with ALS pathogenesis.

The discovery of PFN1 mutations offers us great opportunity to develop new ALS animal models that would open new areas to be explored and to advance the field by elucidating pathological mechanisms and discovering effective therapies for ALS. As such, our approach for developing a new mouse model for ALS is based on the novel human ALS-linked mutations in the PFN1 gene (five point mutations found in fALS patients to date). We have produced transgenic mice using microinjection of vector with mutant and wild-type hPFN1 cDNA constructs. Our PFN1 transgenic construct is produced by using hPFN1 cDNA with G118V mutation engineered and placed in front of mouse prion promoter. Among the four ALS-causing PFN1 mutations identified, G118V mutation was found to be the one inducing a significant decrease in axon growth. We have successfully generated 3 founder mice with human PFN1^{G118V} transgene and the preliminary data from these founders and their progeny demonstrate motor deficits resembling ALS, which appear to occur in a manner dependent upon the dose of mutant PFN1 expressed in the different transgenic strains. F1 progeny display the same signs and symptoms observed in founders, confirming that ALS-like phenotypes are reproducible from generation to generation. This model provides a novel tool to study how hPFN1 mutations cause the development of cardinal phenotypes and pathologies resembling human ALS. This model will be suitable for preclinical studies designed to develop therapeutic strategies to treat or prevent this devastating disease. Novel strategies may include repair of the neuronal cytoskeleton, thereby blocking motor neuron degeneration and reducing axonal pathology and alterations in mitochondrial transport.

All of the current models for ALS have their limitations and each model is a tool to study a specific pathway in the disease, hence the urgency and need for new mouse models to study the PFN1 linked pathway. We have determined that our transgenic mice overexpress hPFN1^{G118V} protein at levels ranging from 1.1- to 8.9-fold higher than endogenous mouse PFN1. We have observed a range of motor deficits resembling ALS, where the highest expressing line has earliest age of onset of symptoms that pass from founder to F1s. We have begun to see the F1 progeny from the highest expressing line have a short life span as they only live 6-8 weeks after onset and start to die by 179 days of age. These results indicate that our new mutant hPFN1^{G118V} transgenic mouse is a viable model and will be highly informative as a mouse model for profilin1 linked ALS and may provide insight for sporadic ALS too.

Existing animal models have contributed to understanding ALS. They have not yet led to effective therapies. It is not clear exactly why these models have not resulted in clinical success, and it is not known what preclinical criteria in the design of new animal models would result in clinical translation. However, generation of new animal models based on different genes and mutations involved in ALS will offer additional opportunities for clinical advancement and for understanding the disease mechanisms. The critical role of PFN1 in actin polymerization, growth cone, and axonal growth is our rationale for generating this viable mouse model for ALS using PFN1 mutations. Such animal models for each gene linked to fALS can potentially be used in advancing our knowledge of disease mechanism(s), and in translating that knowledge to efficacious therapies. Due to similar disease symptoms and pathologies in sporadic ALS (sALS) our PFN1 models likely will facilitate research to understand sALS.

We have remarkably strong evidence that PFN1 mutant transgenic mice, which we have produced, will make significant contributions to ALS research, thereby fulfilling the need for mouse models studying PFN1 neurotoxicity. This mouse model will fill in the knowledge gap in understanding the mechanism(s) underlying motor neuron degeneration associated with ALS. This model will serve as a test platform for developing effective interventions for PFN1 linked ALS patients with the potential to be applicable to a wider spectrum of ALS patients.

### Example 1. Generation of hPFN1 Transgenic Mice

To determine which fALS PFN1 mutation to use for this study, we generated the predicted X-ray crystallographic structure of human PFN1 bound to actin (FIG. 1). The location of the original four PFN1 mutations identified in fALS are indicated and demonstrate the proximal location of the G118V mutation to the actin binding site of hPFN1. We designed transgenic constructs of hPFN1 cDNA with the glycine>valine mutation at amino acid 118 (hPFN1^{G118V}) in front of the mouse prion promoter and wild type hPFN1 cDNA as control (hPFN1^{wild-type}). We contracted with NorClone, Inc. (Ontario, Canada) to make the plasmid construct for human PFN1 containing the G118V mutation. In this construct, we utilized a mouse prion promoter, a promoter which is highly expressed in the CNS and which has been used previously to develop multiple transgenic mouse models of neurodegeneration (Cooper et al., 1998, Schilling et al., 1999, Kiaei et al., 2007, Wegorzewska et al., 2009, Esmaeili et al., 2013). Further, the CNS-specific promoter will avoid pathologies outside the CNS inconsistent with ALS. Following the confirmation of the hPFN1^{G118V} mutation by sequencing, this plasmid vector was microinjected into C57Bl6 mouse fertilized eggs to generate the founder mice.

### Example 2. Three Mutant Human PFN1^{G118V} Transgenic Founder Lines Expressing High, Medium, or Low Copy Number of the Mutant Gene were Established

We created a mouse overexpressing mutant PFN1˜9 fold in the spinal cord which began showing an ALS-like phenotype at 5 months of age and rapidly progressed to full hind and forelimb paralysis and death around 6 months after birth.

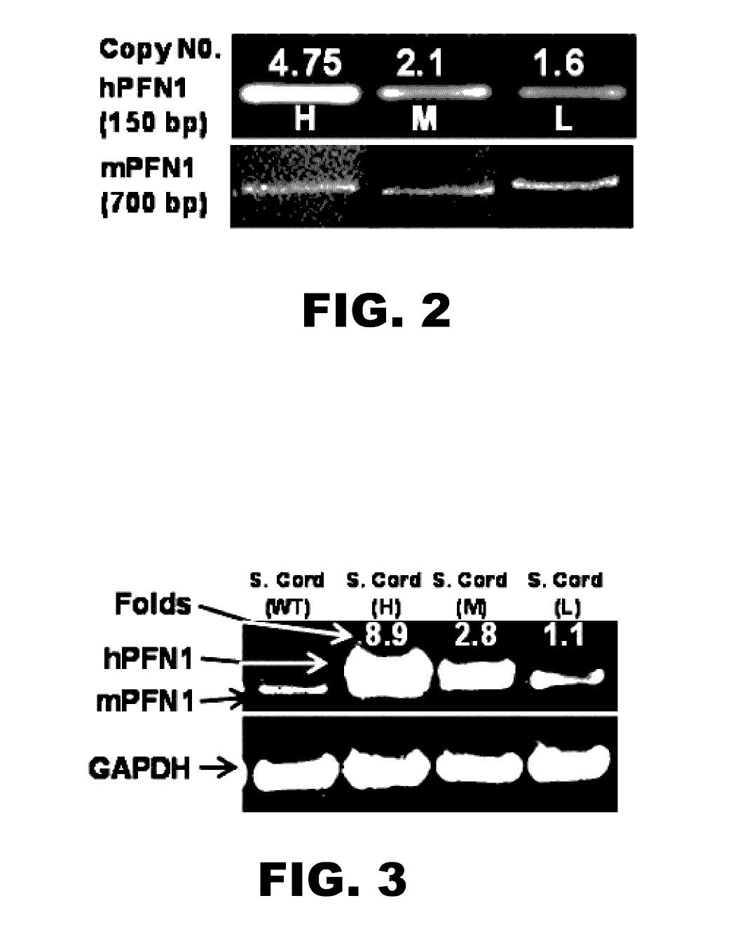

PCR amplification of human PFN1 utilizing tail DNA from the three founders demonstrated that one founder expressed a relatively high copy number of the transgene (designated Founder H) while the two other founders expressed medium (M) or low (L) copy numbers of the human transgene (FIG. 2). The levels of mouse PFN1 DNA were determined to be equal in all founder lines (H, M, and L), as expected (FIG. 2).

The expression of hPFN1 protein expression was evaluated in the H, M, and L founder mice as 8.9, 2.8 and 1.1 fold, respectively. Western blot analysis of spinal cord demonstrated that the hPFN1 protein was expressed at high, medium or low levels corresponding to the level of transgene in the respective founder lines (FIG. 3). Note that hPFN1 migrates slightly slower than mPFN1 in denaturing gels so that a doublet of these proteins is observed in the Western blot. Similarly, as expected, high, medium and low protein expression was observed in brain in the respective founders but, no hPFN1 was detected in liver (data not shown). Wild-type mice expressed mousePFN1 but did not express mutant hPFN1 in any of the tissues evaluated by Western blot (FIG. 3).

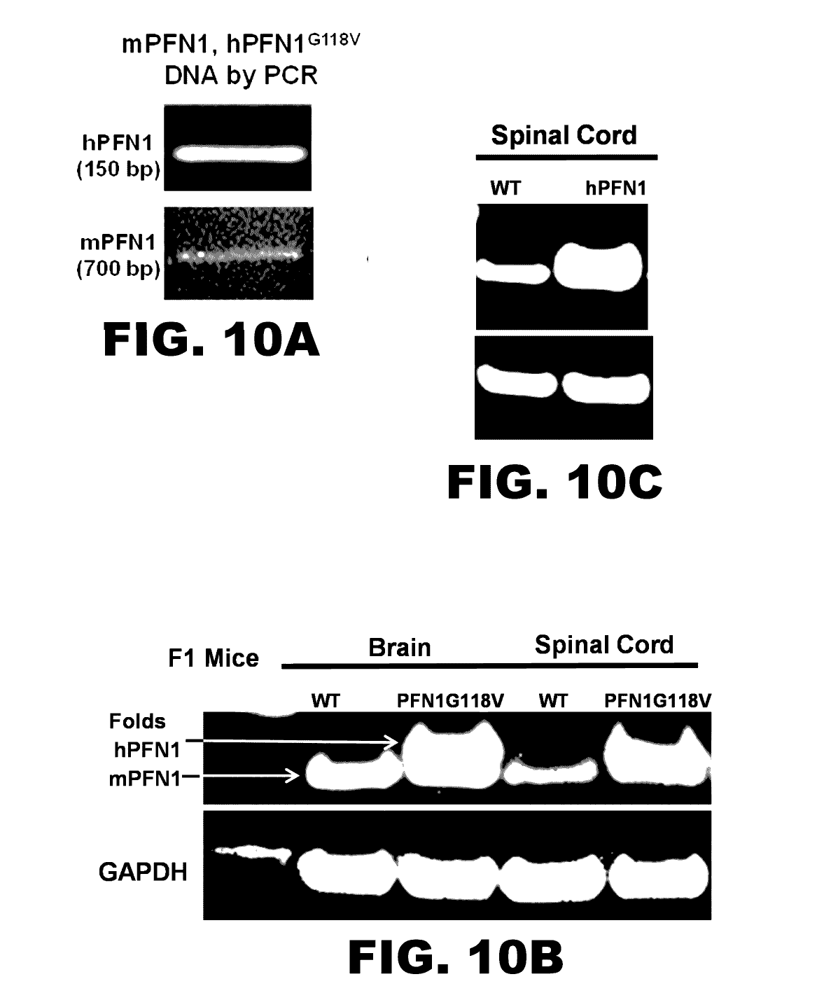

Importantly, the high-expressing Founder H exhibited ALS-like symptoms at 5 months of age. Founders M and L also developed ALS-like symptoms, but the onset of symptoms were displayed from 6-8 months of age. F1 and F2 progeny were produced from the highest expressing founder and the following results are generated in the F1-F2 mice. PCR amplification of human PFN1 from hPFN1^{G118V}mutant F1 mice demonstrated a 3-4 fold increase in copy number of the transgene compared to the mouse endogenous gene (FIG. 10A). Western blot analysis shows high levels of mutant hPFN1^{G118V} in the brain and spinal cord relative to PFN1 endogenous protein. There was no human mutant PFN1 expression in the liver of either hPFN1^{G118V} or non-transgenic mice (data not shown). The mouse endogenous PFN1 protein was present in both transgenic and non-transgenic mice (FIG. 10B). Our human wild-type PFN1 overexpressing transgenic mice show similar expression levels of hPFN1 in the spinal cord (FIG. 10C).

In summary, we have successfully developed three lines of mice with multiple litters of F1 progeny from each Founder. These mice develop ALS-like symptoms, detailed below, and appear to do so in a manner dependent upon the copy number of the hPFN1 transgene expressed by the mice.

### Example 3. ALS-Like Phenotype of Mutant PFN1 Founders and F1 Progeny

Weight Loss:

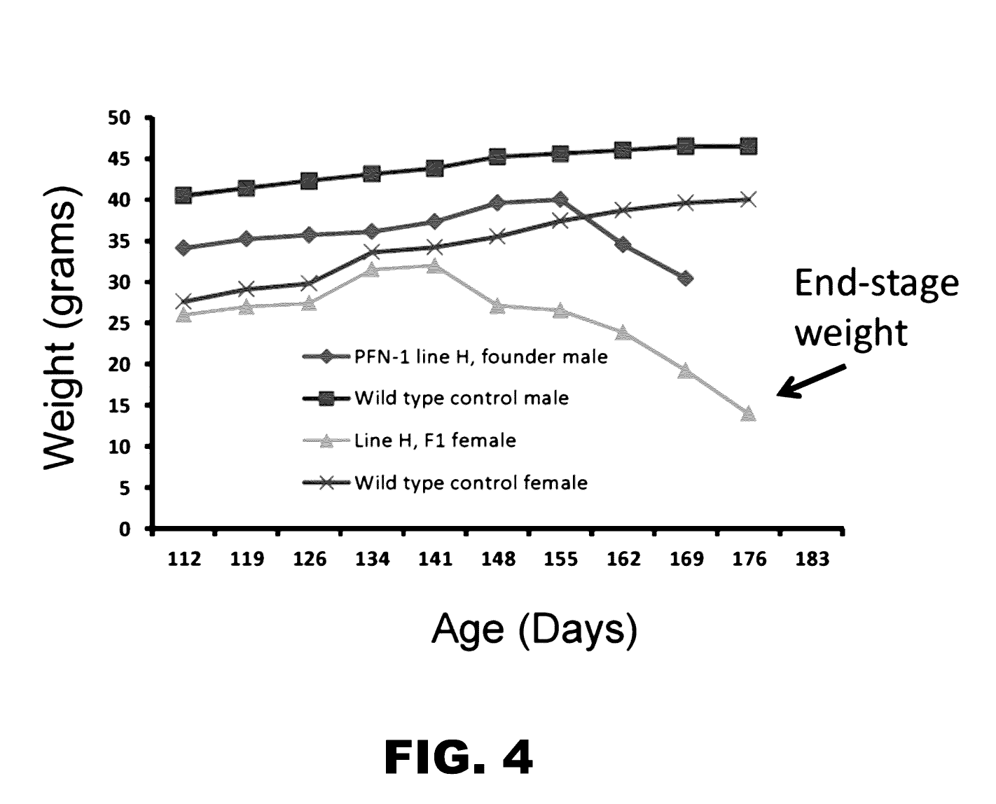

Animal weights were recorded weekly. Wildtype mice demonstrated an expected gradual gain of weight. Founder H and F1 mice also gained weight initially, but then demonstrated a dramatic and rapid loss of weight from 130-140 days of age (FIG. 4).

Neurodegeneration:

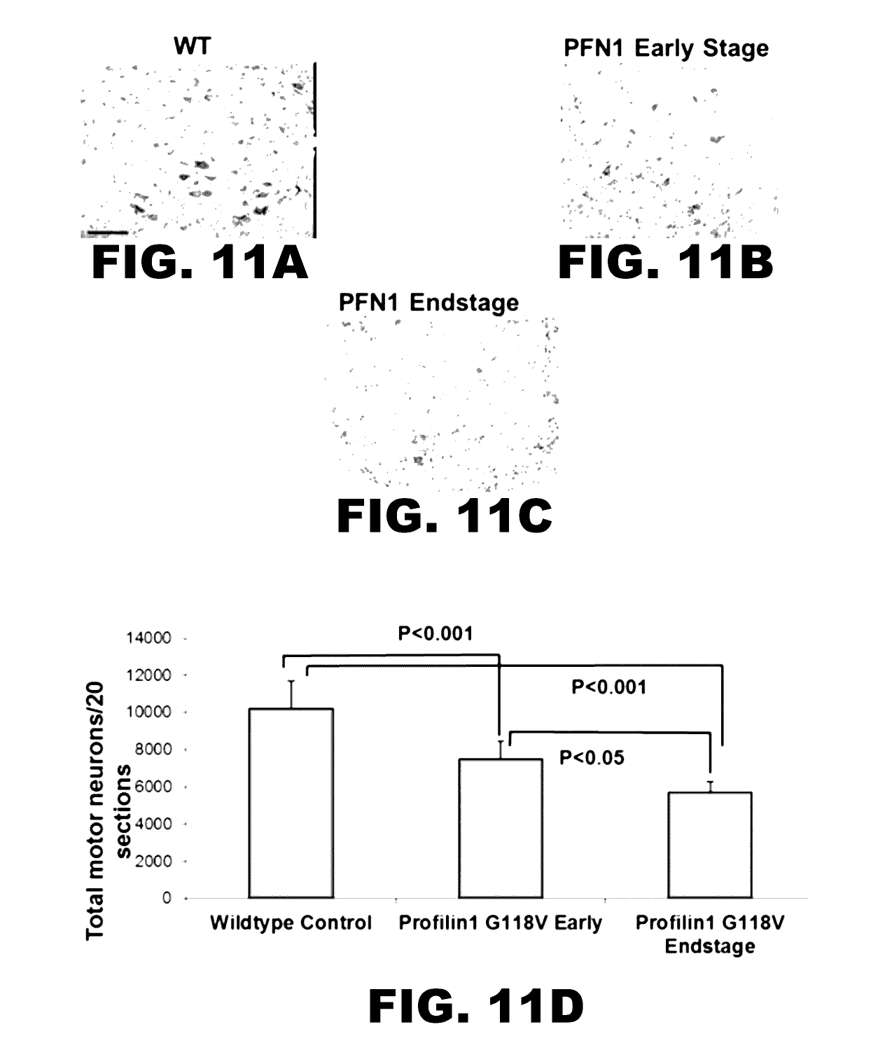

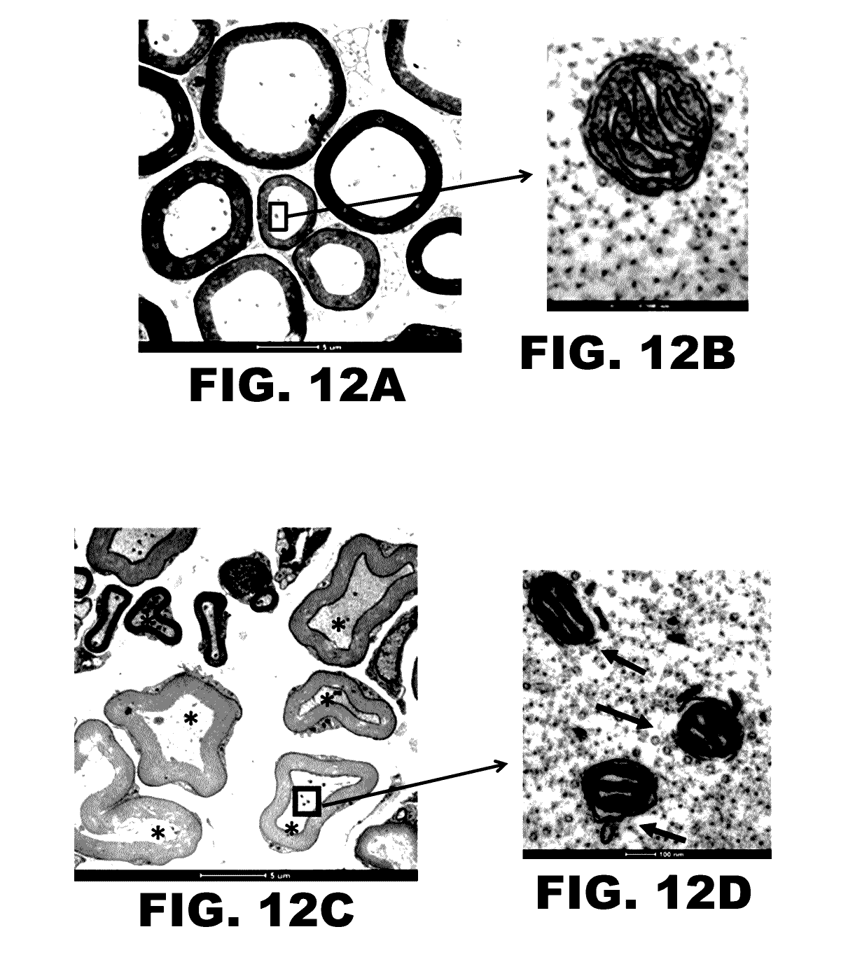

To determine if the ALS-like phenotype in hPFN1^{G118V} mice is associated with neuronal loss in the spinal cord, stereologic cell counts were performed in the lumbar spinal cord of both non-transgenic (wt) and hPFN1^{G118V} mice at 140-178 days of age. Nissl stained neurons in the motor horn were quantified. There was significant loss of neurons in both the early and late stage of the disease in hPFN1^{G118V} mice (FIG. 11A-D). The loss of neurons progressed with time such that late stage disease demonstrated a greater loss of neurons than early stage. Electron microscopy also demonstrated degenerative axonal structure and aberrant mitochondrial structure in hPFN1^{G118V} mice (FIG. 12A-D), evident as membrane blebbing and fragmentation.

Hindlimb Atrophy:

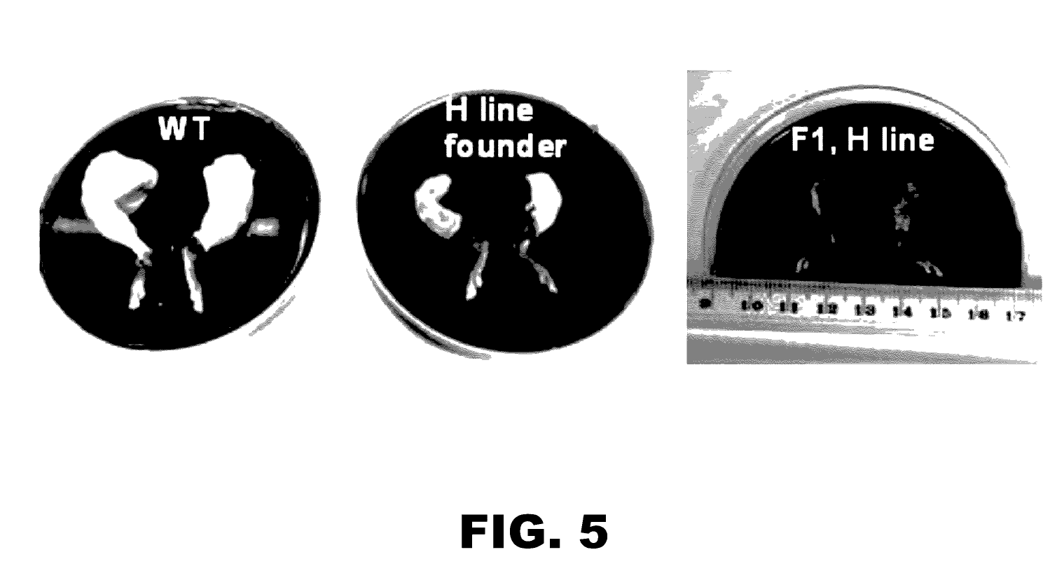

The hPFN1^{G118V} mice exhibited ALS-like motor phenotypes. In contrast to other models of ALS, hPFN1^{G118V} mice have motor function defects in both forelimbs and hindlimbs. hPFN1^{G118V} mice (Founder and F1) exhibited significant skeletal muscle atrophy relative to non-transgenic littermate controls as demonstrated for the hindlimbs (FIG. 5).

Histopathology:

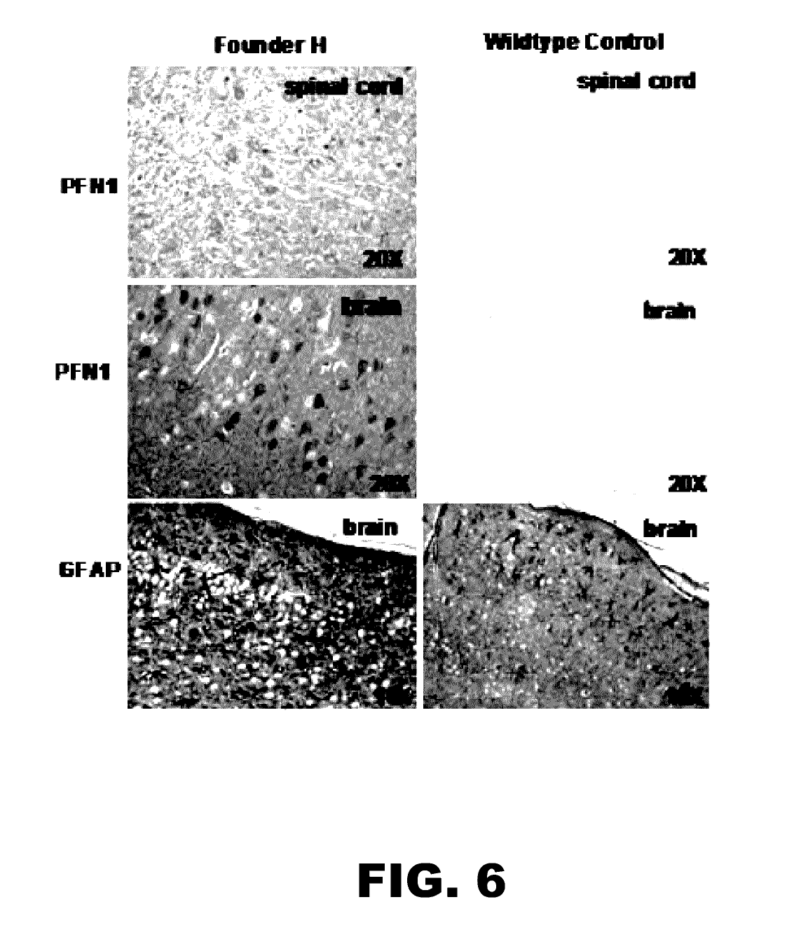

Our initial immunohistochemical analysis (FIG. 6) from Founder H tissues shows over-expression of human PFN1 in brain and spinal cord relative to wild-type controls. Staining with the astrocyte marker (GFAP) revealed astrocytosis.

Behavior:

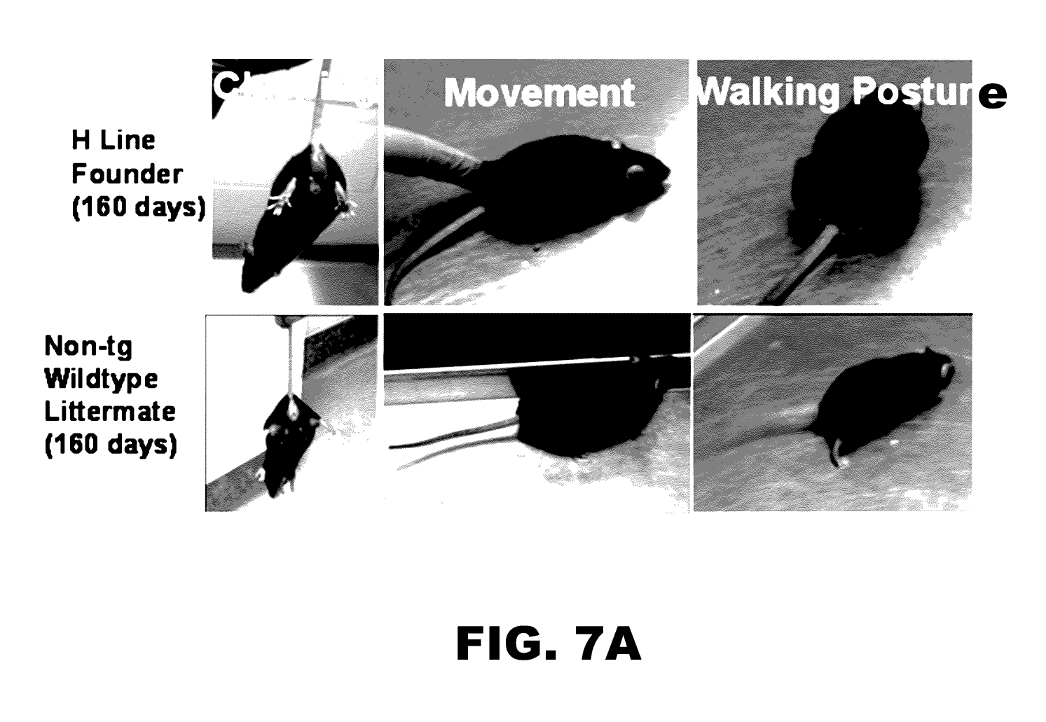

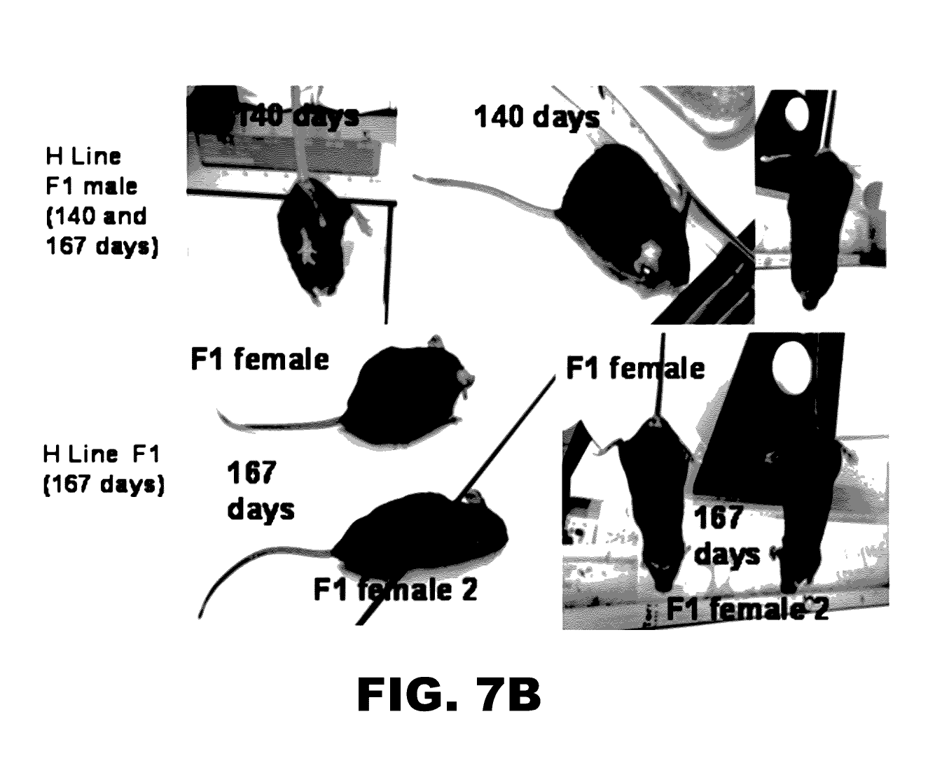

Founder H and F1 mice exhibited ALS-like symptoms at approximately 5 months of age and F1 mice naturally died from ALS at 185 days of age. Each of the hPFN1 transgenic founders (H, M, and L) exhibited ALS-like behavioral phenotypes including hind limb tremor, progressive clasping, atrophy of hind limb skeletal muscles, hind limb muscle weakness, reduced stride length, lower gait, hypokinetic behavior, and reduced motor performance including inability to stay on a rotating rod. We have documented these behavioral changes by video. Although difficult to represent the behavioral impairment of these mice observed by video, we present snap shots of the video to demonstrate some of the behavioral changes in hPFN1 transgenic mice. Using Founder H as an example, we demonstrate that this founder exhibits clasping behavior, reduced movement, and altered walking posture relative to wildtype mice (FIG. 7A). Images of F1 females show hindlimb clasping, abnormal posture, hunched back, low gait (FIG. 7B). Further, hPFN1^{G118V} mice exhibited reduced motor performance compared to non-transgenic mice including impaired performance on a rotating rod (FIG. 14).

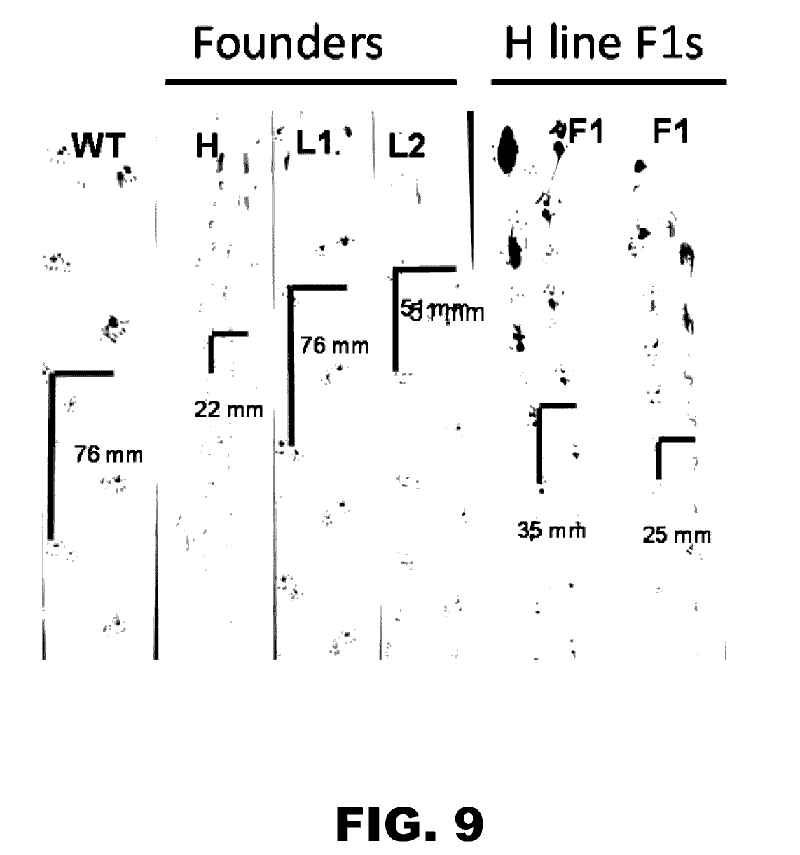

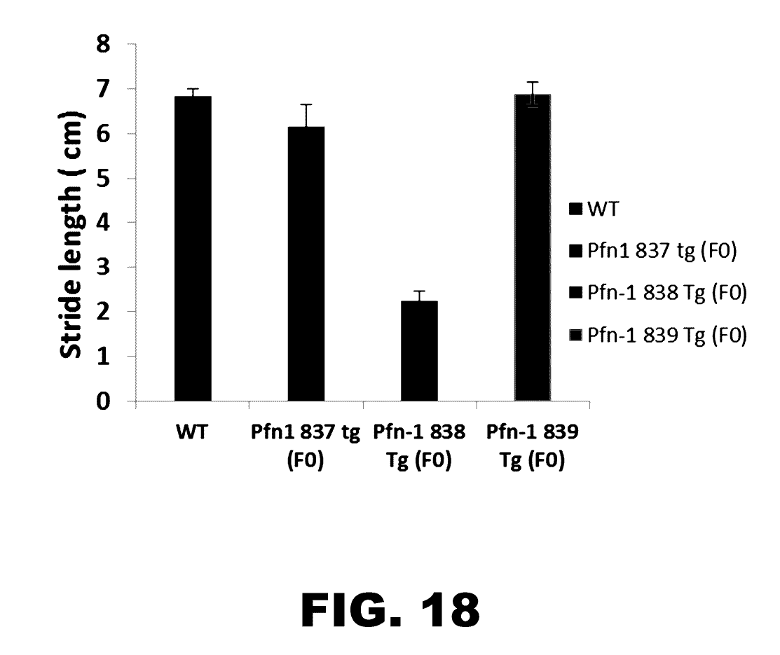

Animals were assessed for stride length and walking pattern by recording their foot prints every week. Initial footprint analysis was carried out using inked paws and analysis of the three founders' stride lengths showed a significant (P<0.05) decrease in hindlimb stride lengths between hPFN1 mutant transgenic mice and control mice (FIG. 9). Interestingly, the stride length of the Founder H was less than those of M and L founders, suggesting that higher transgene expression has a stronger effect on stride length; and possibly on other ALS-like phenotypes. F1 progeny from H line were also assessed and confirmed to have drastic stride length reductions (FIG. 9). FIG. 18 graphically depicts the stride length of WT and the three founders (Founder H, Founder M and Founder L).

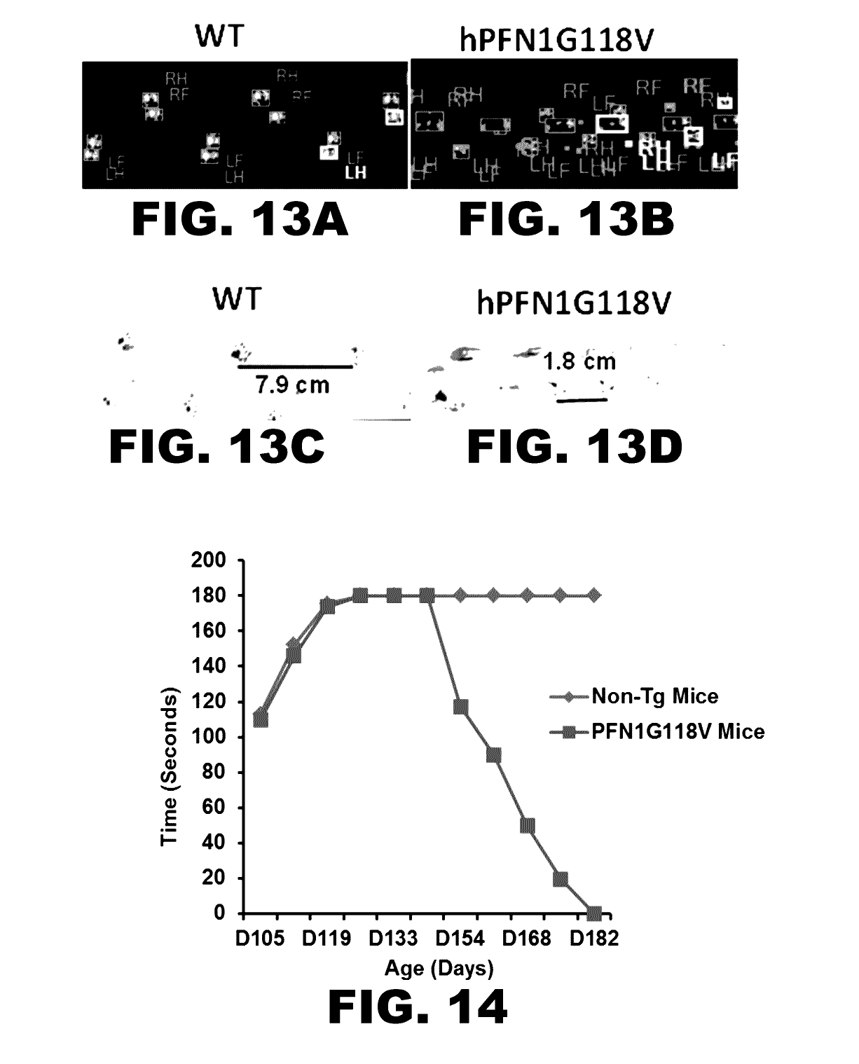

Gait parameters of F1 mice were also assessed weekly utilizing the Noldus CatWalk®. The CatWalk measured almost 200 gait parameters, including both static and dynamic. It documented specific gait parameters that were abnormal in hPFN1^{G118V} mice compared to non-transgenic mice including paw, step sequence, base of support, phase dispersion, and girdle parameters (FIG. 13A-B and Table 1). Inked foot-printing also revealed that hPFN1^{G118V} mice had abnormal gait (FIG. 13C-D).

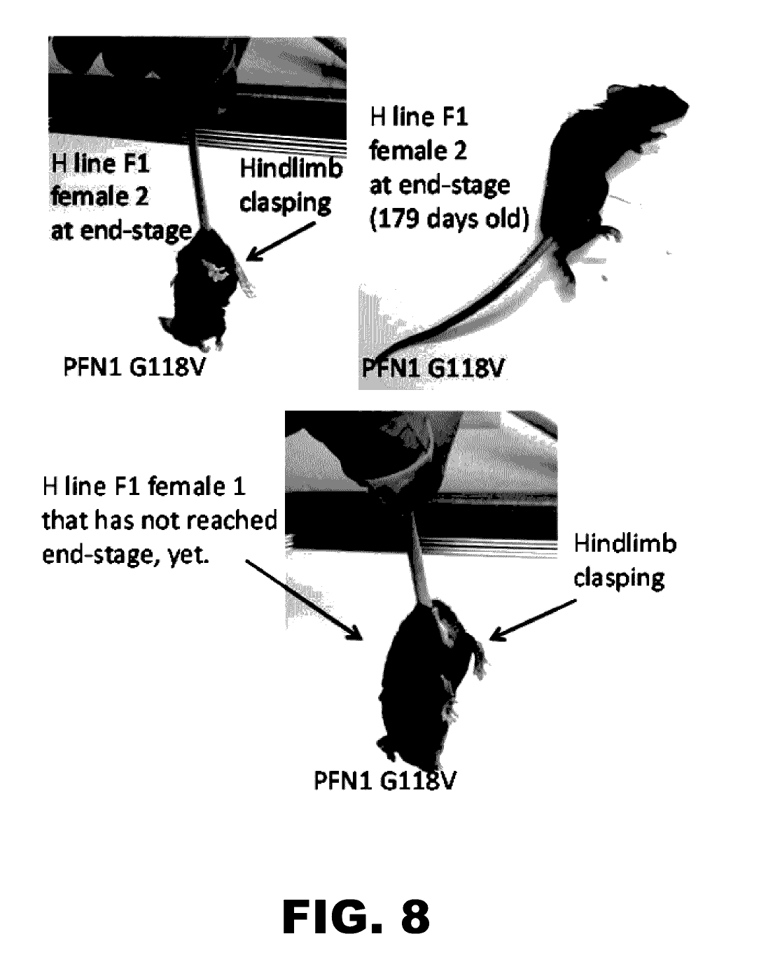

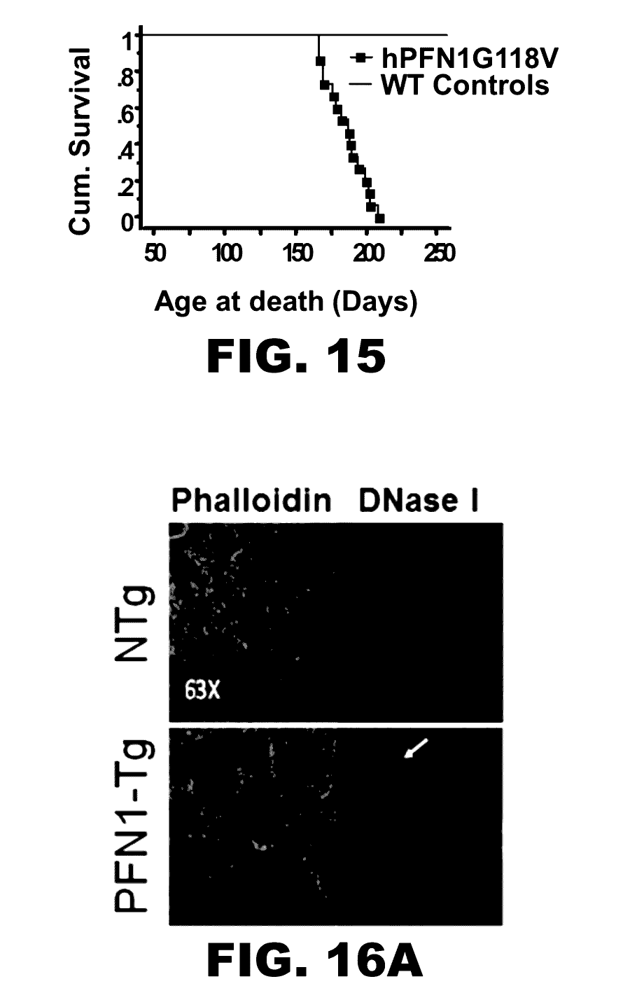

Additionally, we have obtained dramatic data on the F1 progeny from hPFN1^{G118V} high expressing line. One female F1 reached the end-stage and died at 179 days of age and another will likely reach the end-stage in 7-10 days following the date of the image (FIG. 8). As graphically depicted, survival of hPFN1^{G118V} mice was dramatically reduced compared to non-transgenic mice due to progressive voluntary muscle paralysis (FIG. 15).

TABLE 1
CatWalk analysis reveals hPFN1^{G118V} mice have
gait abnormalities compared to wt controls.

| GAIT PARAMETER | Ratio: hPFN1^{G118V}/wt |
|---|---|
| Paw (LH) | |
| Print Area | 0.49* |
| Intensity | 0.83** |
| Stride Length | 0.50*** |
| Step Sequence | |
| Regularity Index | 0.88* |
| Base of Support | |
| Hind Paws | 0.48* |
| Diagonal Phase Dispersion | |
| RF- LH | 2.2* |
| Girdle | |
| LH-RH | 0.80** |

Footnotes:
*p < 0.05,
**p < 0.01,
***p < 0.001
R = right,
L = left,
F = front,
H = hind

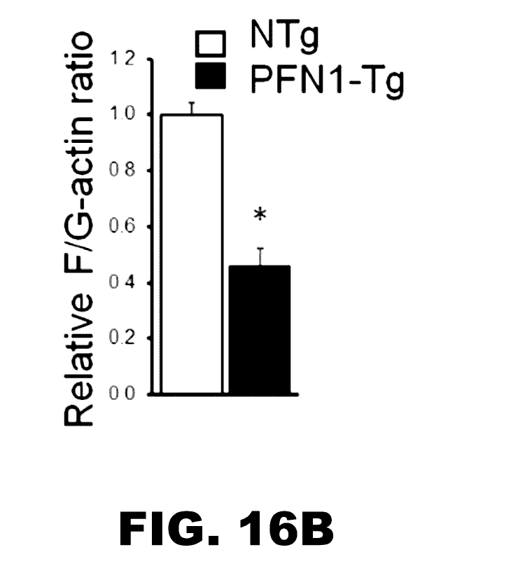

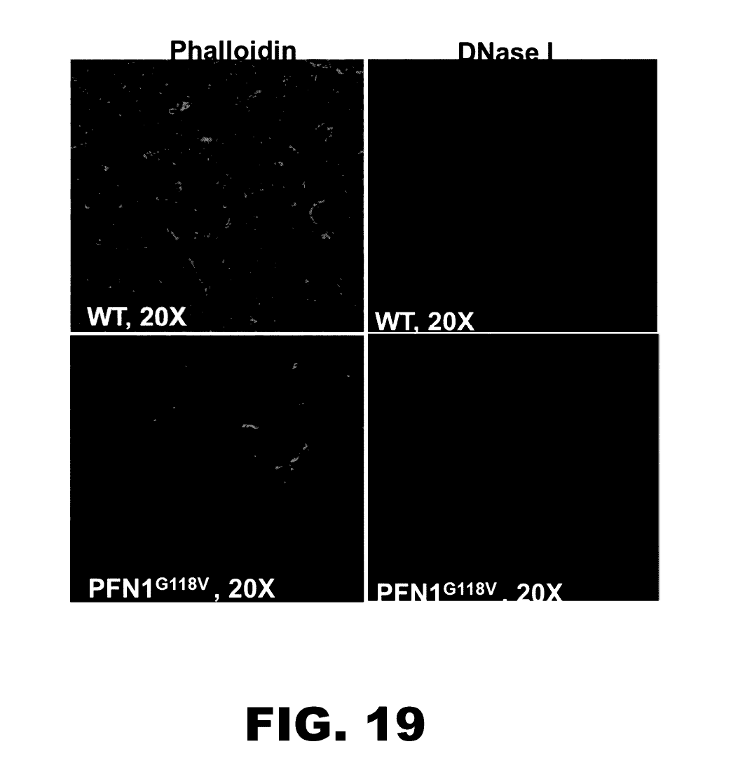

Mutant PFN1 transfected PMNs have significant decreases in axon growth, growth cone size and altered growth cone morphology. PFN1 mutant toxicity is suggested to affect the F/G ratio. PMNs transfected with two mutations (C71G and G118V) found in ALS patients and one synthetic mutation (H120E) have lower F/G ratios. Here, we show preliminary in vivo data from lumbar spinal cord of fully symptomatic hPFN1^{G118V} mice which demonstrate a reduced F/G ratio relative to wild type mice (FIG. 16A-B and FIG. 19). This suggests that mutant PFN1 may cause dis-regulation of actin polymerization.

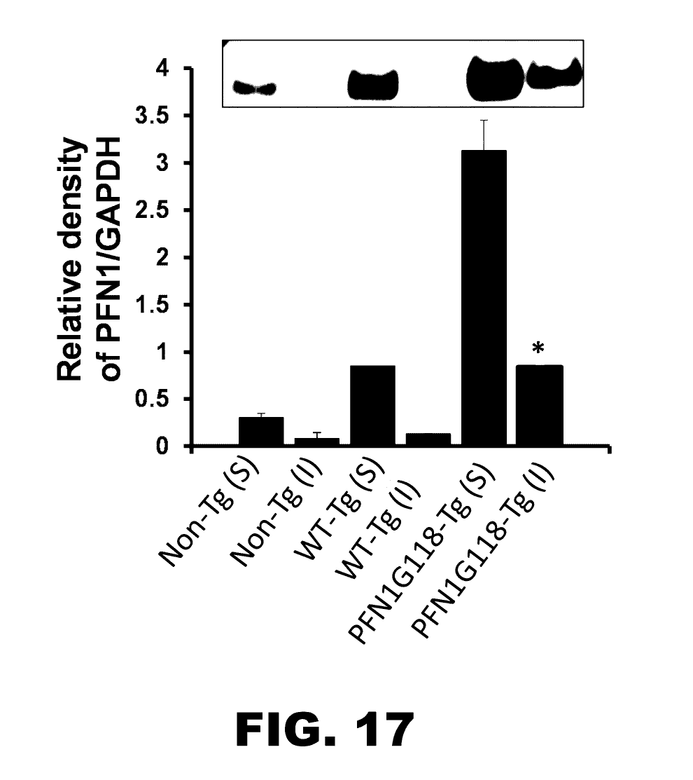

Profilin1 mutants (PFN1^{C71G}, PFN1^{M114T}, PFN1^{G118V}) were found in the insoluble aggregates isolated from transfected PMNs. Now, we show mutant PFN1^{G118V} exists in the insoluble fractions derived from total homogenates from the spinal cord of mutant PFN1^{G118V} transgenic mice (FIG. 17). This confirms the in vitro data and further suggests that aggregation of mutant PFN1 may contribute to neurotoxicity in ALS. In this study, we will investigate this in more detail by examining brain and spinal cord tissues from pre-onset, onset, symptomatic and end-stage PFN1^{G118V} mutant mice.

Collectively, these studies demonstrate that the hPFN1^{G118V} mouse model expresses hallmark features of the phenotype and pathology of human ALS-like disease. This suggests that further characterization of the model is important and that investigation of the cellular and molecular mechanisms of mutant hPFN1^{G118V} activity may reveal yet unidentified mechanisms of ALS pathogenesis. Our results suggest that mutations in PFN1 may contribute to ALS-like disease pathogenicity in part by altering axon dynamics. The classical function of PFN1 in actin polymerization is disrupted which may result in axonal cytoskeletal disruption. These preliminary data need to be further verified with mutant and wild type hPFN1 overexpressing mice.

### Example 4. Functional Phenotype and Pathology of Mutant hPFN1^{G118V} Transgenic Mice Resemble that of ALS Patients

We hypothesize that expression of hPFN1^{G118V} mutant protein in mice will cause cardinal phenotypes and pathologies that resemble those in ALS patients. Despite decades of research we still do not fully understand the cellular and molecular mechanisms of pathogenesis of ALS that lead to motor neuron degeneration. As a result, viable therapeutic strategies for ALS are lacking. Better understanding would be facilitated by investigation of new models of ALS that would have the potential to identify novel pathways, or pathways that would coordinate with known pathways of neurodegeneration. To address this critical need, we developed a transgenic mouse overexpressing the human PFN1^{G118V} mutation, one of five PFN1 mutations that were recently identified in some fALS patients. Mutation of amino acid 118 was selected because it is an important site proximal to the actin binding site in PFN1 that is essential to PFN1 function and neuron survival. The fALS mutations in PFN1 have only begun to be studied in primary motor neuron cultures and in vivo studies have not been possible due to the lack of a mouse model of PFN1 mutation. Characterization of ALS-like disease phenotype and pathology in the hPFN1^{G118V} mouse, as described above, suggests that it is a robust new model for study of ALS. For a model to be relevant for study of PFN1-linked ALS, it must recapitulate the behavioral phenotype and the pathology of ALS. Thus, we propose to further characterize the ALS functional phenotype and pathology in the brain, spinal cord, and skeletal muscle to better characterize this mouse as a valuable tool for identification and investigation of novel cellular and molecular mechanisms of pathogenesis in ALS.

Functional Phenotype:

Individuals with ALS suffer from rapidly progressing voluntary muscle deterioration, paralysis, weight loss, and premature death. This clinically-relevant ALS phenotype must be documented in the hPFN1^{G118V} mouse if it is to serve as a suitable model for new investigations into ALS. Accordingly, key functional phenotypic endpoints will be assessed and quantified in these mice including muscle weakness, muscle atrophy, paralysis, motor dysfunction (specifically gait, balance, tremor, clasping, tail drooping, and hunched back), hypokinesis, progression of weight loss, and premature death. Analyses will be conducted longitudinally at non-symptomatic, symptomatic, and end-stage disease time points. Control comparisons will include wild type non-transgenic control mice and transgenic mice overexpressing wild type human PFN1. Paresis to paralysis will be assessed by hindlimb appearance, clasping, activity level, automated CatWalk, and rotarod. Tremor, clasping, tail drooping, and hunched back will be assessed^{22,25}. Hypokinesis will be quantified by the automated EthoVision system, which is a video software system to track the movements and activity of mice. Gait will be quantified by analysis of both dynamic and static parameters of paw, step sequence, base of support, phase dispersion, and girdle in early and late stage disease using the automated CatWalk® system. Balance and motor coordination will be quantified by analysis of walking ability on a rotating rod (rotarod) biweekly beginning on postnatal day 60. The time to first fall and the number of falls will be recorded. Weight will be measured weekly beginning at postnatal day 30. The age at which the animal becomes moribund (inability to right within 20 seconds) will be recorded. Muscle atrophy will be assessed by determination of the weight of limb skeletal muscle. Forelimb strength will be assessed by grip strength²⁵. All of the behavioral tests will be performed on all of the mice in the order noted above. The resulting data will provide a comprehensive, quantitative and qualitative assessment of the ALS-like disease phenotypes.

Pathology:

Individuals with ALS exhibit specific pathology in the neuromuscular axis that includes loss of motor neurons in the brain and spinal cord, degeneration of axons, denervation of neuromuscular junctions, degeneration of skeletal muscle, glial activation, and aggregation of specific proteins. Each of these endpoints will be assessed quantitatively in the hPFN1^{G118V} mutant mice, non-transgenic control mice, and transgenic mice overexpressing wild type human PFN1. Analyses will be conducted longitudinally at non-symptomatic, symptomatic, and end-stage disease time points in tissue sections^{22,25,26}. Stereological cell counts of neurons in the motor cortex and spinal cord will be performed²⁶. Presence of PFN1 in synaptic sites and neuromuscular junctions will be examined. Degeneration of skeletal muscle will be quantified by muscle weight and determination of fiber type by immunohistochemistry using anti-mATPase and anti-succinate dehydrogenase antibodies.

Axonal Degeneration and Neuromuscular Junction Disruption: Neuromuscular Junction Immunohistochemistry:

Motor weakness (FIG. 14), progressive hindlimb clasping, impaired gait (FIG. 13) due to nerve and muscle degeneration and hind limb muscle atrophy are evident in PFN1^{G118V} mice (FIG. 5). It is possible that the neuromuscular junction is disrupted. Therefore, we will assess neuromuscular junctions with fluorescently labeled α-bungarotoxin, and antibodies to neurofilament or acetylcholine esterase^{27,28}. To determine the time that NMJ degeneration starts and how extensively it progresses during disease development, we will examine hind and forelimb muscles at pre-onset, onset, fully symptomatic and end stage of disease. We will also examine electrophysiological abnormalities by electromyography (EMG) and motor unit function using motor unit number estimation (MUNE) techniques that are being used for quantifying motor unit function in living patients. MUNE has proved useful in predicting rate of progression and survival of ALS²⁹. Motor end plate will be visualized using fluorescent-conjugated α-bungarotoxin, which bind irreversibly to post-synaptic acetylcholine receptors on the skeletal muscle plasma membrane as described²⁸.

Quantitative Analysis of Neuromuscular Junctions and Ventral Roots:

For neuromuscular junction imaging mice will be perfused first with a PBS prewash for 2 mins followed by 4% PFA in PBS for 5 mins. Gastrocnemius and tibialis anterior muscles are dissected and post-fixed overnight in 1.5% PFA at 4° C. Muscles are washed in PBS, incubated in 25% sucrose in PBS overnight at 4° C. and embedded in OCT (Thermo). 35 um cryosections are collected, mounted on glass slides and stored at −80° C. until use. Frozen sections are air dried for 30 mins and washed with PBS, followed by blocking in 10% goat-serum in 10% tritonX₁₀₀ for 3 hrs. Sections are incubated for 24 hrs at 4° C. in a primary antibody solution containing a cocktail of rabbit anti-synaptophysin (1:5, Invitrogen) and rabbit anti-Neuronal Class III Beta-Tubulin (1:1000, Covance) diluted in blocking solution. Following washing sections are incubated in PBS containing secondary antibodies alexa-488 conjugated donkey anti-Rabbit (1:500) and alexa-555 conjugated alpha-bungarotoxin (1:500) overnight at 4° C. Sections are washed, counterstained with DAPI and coverslips mounted using Immumount.

Ventral Root Analysis Entails Semi-Thin Sectioning, Toluidine Blue Staining and Axon Quantification.

Mice are fixed by perfusion with 4% PFA in PBS and L5 ventral nerve roots dissected. Following overnight fixation in 2.5% gluteraldehyde in 0.1 M cacodylate buffer nerves are washed and further post-fixed in 1% osmium tetroxide for 1 hr. Samples are dehydrated through a graded ethanol series into propylene oxide, followed by overnight infiltration in 1:1 solution of propylene oxide and SPI-Pon 812 resin mixture. Following 3 hrs of incubation in SPI-Pon 812 resin samples are polymerized at 68° C. for 4 days. Nerves are trimmed, reoriented and 0.6 um semi-thin sections collected. Semi-thin sections were mounted on glass slides, followed incubation in toluidine blue on a hot plate for 30 seconds. Following washing sections are dried and a coverslip mounted using DPX. Sections are imaged and axon number and diameter quantified using ImageJ.

Quantitative Motor Unit Analysis Using Electromyography (EMG), Motor Unit Size (MUS) and Motor Unit Number Estimates (MUNE).

EMG and MUNE have proved useful in quantifying the status of the motor unit and in predicting the rate of progression and survival in SOD1G93A mice²⁹. In the proposed studies, we will perform quantitative EMG analysis on hPFN1-WT vs hPFN1G118V mice. We will compare MUS and MUNE in these two groups of mice (n=5 for each group) using EMG at 60, 90, and 120 days. Briefly, after animals are anesthetized, the cathode of the stimulating electrode is positioned at the sciatic nerve in the thigh while a subcutaneous anode is placed 1 cm proximally. Motor responses are recorded from a surface electrode located circumferentially around the hind limb (recording activity in both flexor and extensor compartments). The reference electrode is located subcutaneously in the foot. A maximum response reflecting activation of all viable motor axons is recorded. Repeated stimuli are then applied at very low intensities, slowly increasing the intensity until a single all-or-none response is obtained, reflecting the lowest threshold single motor unit. Intensity is slowly increased until at least 10 well-defined increments are recorded. Digital subtraction of successive increments yields the individual motor units. The average motor unit amplitude (average motor unit size or MUS) is then divided into the maximum response size to yielded the motor unit number estimate (MUNE). For statistical analysis, the effect of each genotype on MUS and MUNE are analyzed using a mixed model analysis of variance.

It will be important in future studies to investigate if there is an ALS phenotype in (a) a knockin human or mouse PFN1 transgenic mouse, and (b) a transgenic mouse overexpressing the mutant human or mouse PFN1 gene driven by the endogenous profilin promoter. As noted above, we have also produced wild-type hPFN1 overexpressing mice as controls. These mice overexpress human wild-type PFN1 at similar levels to hPFN1^{G118V} line. A founder overexpressing hPFN1 has lived over six months and is healthy. The current hPFN1^{G118V} overexpressing mouse and the series of experiments we propose to use in this study are designed to provide proof-of-principle that mutation of the PFN1 gene can produce an ALS phenotype and pathology and provide a model to begin the investigation of the mechanism of neurotoxicity mediated by the mutant PFN1 gene. This model will allow study of cytoskeletal defects and the impact of PFN1 mutation on interactions with actin and other PFN1 partners. This study will result in a fully developed and characterized mouse model for ALS with the PFN1 mutation. Our model will serve to shape the basis of mechanistic studies on mutant PFN1 mediated cytoskeletal disruption. Other models such as knockin models may be developed to test the effect of PFN1 mutation if driven by the endogenous promoter in the future. Future plans will also include therapeutic testing of new compounds in the hPFN1^{G118V} model. It is noted that wild-type PFN1 expression driven by the mouse smooth muscle α-actin promoter resulted in increased expression of PFN1 in vascular tissue, vascular hypertrophy, and hypertension^{34,35}. The promoter we have utilized is not expected to direct expression of PFN1 to vascular tissues because it is CNS-specific.

### Example 5. Mechanisms of Mutant hPFN1^{G118V} Activity that Underlie Degeneration of Motor Neurons

We hypothesize that expression of hPFN1^{G118V} mutant protein in mice will cause neurodegeneration. It is not known how PFN1 mutations result in the phenotype and pathology of ALS. However, it is important to gain this knowledge because discovery of the cellular and molecular mechanisms of mutant PFN1 activity is clearly relevant to the subset of fALS patients carrying PFN1 mutations but is also relevant to other ALS patients because the identified mechanisms may represent pathogenic mechanisms involved in sporadic ALS. To address this need, we will use cultures of primary spinal cord motor neurons, homogenates of spinal cord tissue, and tissue sections from the spinal cord to investigate the mechanisms of hPFN1^{G118V} activity. This investigation will focus on discovery of new pathogenic mechanisms based on mutant PFN1 interaction with actin and regulation of cytoskeletal function. In all experiments, hPFN1^{G118V} mutant mice, non-transgenic control mice, and transgenic mice overexpressing wild type human PFN1 will be compared at pre-onset, onset, fully symptomatic, and end stage disease. Primary cultures of spinal cord motor neurons will be utilized to quantify the impact of the hPFN1^{G118V} mutation on motor neuron viability, growth cone formation, neurite formation, cytoskeletal structure, and mitochondrial structure. These endpoints are well suited for analysis in primary motor neuron cultures³⁶. Neuronal viability will be quantified with the live-dead assay (Molecular Probe/Invitrogen, Carlsbad, Calif.). Growth cone formation will be quantified by growth associated protein-43 (GAP-43) immunohistochemistry. Neurite formation will be quantified by membrane associated protein-2 (MAP-2) immunohistochemistry. The integrity of the cytoskeletal structure will be assessed quantitatively and qualitatively by phalloidin actin staining. All immunohistochemistry will be quantified using MetaMorph software (Molecular Devices, Sunnyvale, Calif.).

Since we have observed mitochondrial membrane damage in ventral root axons from fully symptomatic PFN1 mutant mice, we will further study mitochondrial structural integrity and function qualitatively and quantitatively by biochemical assays and by electron microscopy using mitochondria from mouse tissue and cell cultures^{27,37-40}. To access mitochondrial integrity and function, we will study: a) Structure, morphology and localization. We will examine structural damage on mitochondria in brain, spinal cord and skeletal muscle tissues isolated from PFN1 mutant and wild-type PFN1 mice at pre-onset, onset, fully symptomatic and end stage of disease using light, confocal and electron microscopy. In these in vivo and in vitro studies, markers for mitochondria will be used in immunohistochemical examination to determine cellular distribution and localization. b) Function: 1) We will utilize total brain and pooled spinal cords and/or cultured primary astrocytes to isolate mitochondria to measure the changes in mitochondrial membrane potential using the potentiometric dye TMRM (Invitrogen, Carlsbad, Calif.), as previously described⁴¹. 2) Brain isolated mitochondrial Ca²⁺ handling will be measured using a combination of the Ca²⁺ indicator dyes Fluo-4 AM for cytosolic Ca²⁺ and Rhod-2 AM for mitochondrial Ca²⁺. The combination of these two dyes will provide a comprehensive representation of regional intracellular Ca²⁺ handling. 3) Production of reactive oxygen species (ROS) will be assessed using the fluorescent dyes MitoSOX and H₂DCFDA, which detect ROS in mitochondria or in the whole cell, respectively. MitoSOX, at an excitation wavelength of 405 nm, has the advantage of detecting the level of upstream superoxide generated in mitochondria. This is in dynamic equilibrium with mitochondria superoxide dismutases while H₂DCFDA detects ROS that are generated throughout the cells by the action of multiple downstream reactions. 4) ATP synthesis and oxygen consumption in the presence of different substrates will be assessed in total cell extracts from primary astrocytic cultures using established methods⁴².

Glia can mediate neurodegeneration in ALS^{43,44} and may contribute to expression of the ALS phenotype in hPFN1^{G118V} mice. The contribution of hPFN1^{G118V} mutant astrocytes and microglia to neurodegeneration will be determined by co-culture of hPFN1^{G118V} mutant glia with wild-type motor neurons using viability, neurite and growth cone formation, and axonal length as readouts. In this analysis, spinal cord glia form the early onset, onset, fully symptomatic, and end stage mice and E12.5 day embryos will be established in culture and isolated wild-type spinal cord motor neurons from E12.5 day embryos will be added to the cultures.

Oxidative stress and neuroinflammation have been demonstrated to be important mechanisms contributing to ALS pathogenesis. To determine if these mechanisms are also at play in the hPFN1^{G118V} mouse, the level of oxidative stress will be determined in motor cortex and spinal cord homogenates by quantification of malondialdehyde (lipid peroxidation)^{40,46}, and 8-OHdG (oxidized DNA adduct)⁴⁷. Activation of astrocytes and microglia will be assessed by quantitative morphometry (MetaMorph® software, Molecular Devices, Sunnyvale, Calif.) in the motor cortex and the motor horn of the spinal cord by immunohistochemical staining with GFAP (astrocytes) or CD11b and Iba1 (microglia) antibodies²⁶. Neuroinflammation in the motor cortex and spinal cord will be assessed in homogenates by quantification of inflammatory molecules shown to be increased in humans and SOD1 mice, including TNFα, IL-1β, COX-2, and FasL^{26,48,49} by ELISA (protein) and real time PCR (RNA). Aggregation of TDP-43 and ubiquitination will be assessed in brain and spinal cord tissues by immunohistochemistry and quantitative morphometry^{1,23,24}.

We expect that hPFN1^{G118V} protein will have impaired actin binding and that the integrity of the cytoskeleton and cytoskeletal-mediated activities such as neuron survival, growth cone formation, and neurite extension will be reduced in hPFN1^{G118V} mice. If mitochondrial structure or ATP level is altered in hPFN1^{G118V} mice it will suggest that mitochondrial function is impaired. Alternative sources for mitochondria would be to use Neuro2A, HeLa or COS-7 cell lines to transfect with our already available mutant and wild-type PFN1 constructs and further analyze our finding from in vivo and primary astrocytes. If mutant glia are toxic to wild-type neurons, it will suggests that glial neurotoxicity mechanisms contribute to ALS. If only a subset of the above endpoints test positive, we will consider that some pathogenic mechanisms of ALS identified in other mouse models may not be involved in fALS with the PFN1^{G118V} mutation.

### Example 6. Mechanisms of Mutant hPFN1^{G118V} Activity Leading to Formation of Aggregates and Dysregulation in Actin Polymerization In Vivo and In Vitro

We hypothesize that expression of hPFN1^{G118V} mutant protein in vivo and in vitro results in formation of aggregates, alteration in PFN1 protein-protein interactions, and dysregulation of actin polymerization. This could contribute to the disruption of the axonal cytoskeleton and synapse.

Characterization of Insoluble Aggregates.

Protein aggregation has been shown to be a hallmark of neurodegenerative disorders including ALS. Transfection of PMNs with PFN1 mutants resulted in formation of aggregates containing PFN1, suggesting that these aggregates may contribute to the death of PMNs. In the proposed studies we will determine if PFN1 mutant mice and PMNs derived from them also exhibit PFN1 containing aggregates. Furthermore, we will determine if proteins including TDP-43, are also observed in the mutant PFN1 mice. We will also use an unbiased proteomic approach to identify additional protein(s) found in these insoluble aggregates.

Alterations of PFN1 Protein-Protein Interactions and Dysregulation of Actin Polymerization.

PFN1 protein alterations inhibit neurite outgrowth^{50,51}. The presence of PFN1 mutations (C71G, M114T, and G118V), significantly decreased axon outgrowth. This suggests that mutations in PFN1 may contribute to ALS pathogenicity in part by inhibiting axon dynamics. Defective actin dynamics in mutant PFN1 mice and PMNs, including PFN1 interaction with its binding partners, and its actin-polymerizing activity both in vitro and in vivo will be investigated. The objective is to identify how mutations in PFN1 alter its normal cellular function, impairing downstream actin-dependent pathways and eventually leading to motor neuron degeneration. Our working hypothesis is that mutations in PFN1 diminish its actin polymerizing activity rendering neurons and their cytoskeletal processes with reduced F-actin and accumulation of G-actin. Mutation in PFN1, such as G118V, may also alter its interaction with several binding partners, either directly or through sequestration into insoluble aggregates. The rationale is that our mutant PFN1 mice exhibit motor neurons and ventral horn axonal roots degeneration, and we see reduced F-actin and increased G-actin, in the spinal cord of these mice. This research will contribute to understanding the molecular basis of PFN1-linked ALS. Such knowledge will increase our understanding of disease pathogenesis and may lead to targeted therapeutic approaches.

The Effect of Mutant hPFN1^{G118V} Activity Leading to Insoluble PFN1 and Aggregation In Vivo and In Vitro.

We previously found evidence for the presence of PFN1 in insoluble fractions. Here, we demonstrate the presence of mutant PFN1^{G118V} in spinal cord homogenates derived NP-40 insoluble fractions in vivo. We will expand the study and further examine the presence of insoluble PFN1 in mutant PFN1 mice. Therefore we will examine spinal cord, and brain total protein extracts for NP-40 soluble/insoluble fractions and investigate the extent of PFN1 insolubility during disease development and progression from pre-onset to end stage of disease. We will further investigate the NP-40 insoluble fractions by proteomic analysis to identify novel aggregating proteins.

Soluble/Insoluble Assay:

To test the aggregation of PFN1's cellular partners by NP40-insoluble/soluble cellular fractionation, brain and spinal cord from PFN1^{G118V} and wild-type PFN1 mice at pre-onset, onset, fully symptomatic and end-stage of disease will be analyzed. In addition, in vitro studies will be performed using PMNs from wild-type and mutant E12.5 PFN1 transgenic mice. These cells will be cultured and analyzed for localization of insoluble proteins. PMNs will also be transfected with either V5-tagged WT or mutant PFN1^{G118V}. Detergent-soluble and insoluble protein fractions isolated from transfected cells will be prepared as described above¹ and subjected to western blotting using antibodies directed against PFN1 and TDP-43. We will also perform unbiased proteomics to define the components of aggregates found in cells and tissues overexpressing mutant PFN1.

The Effect of Mutant hPFN1^{g1181} Activity on PFN1 Protein-Protein Interaction and Resulting Alterations in Neuronal Cytoskeleton, Axons, and Synapses.

The regulation of the actin cytoskeleton through the activity of PFN1 and other actin-binding proteins has long been considered to be crucial in the maintenance of cellular structures in both developing and mature neurons. Our previous studies demonstrated that pathogenic mutations in PFN1 reduce its affinity for actin, and result in a reduction in F-actin compared to G-actin levels in growth cones¹ However, we have yet to establish whether mutations in PFN1 influence its ability to bind other known binding partners involved in actin dynamics. We will investigate PFN1 interaction with well-known neuronal relevant binding partners^{2,13,16}. Although PFN1 has been shown to interact with more than 50 proteins¹⁶, we will focus our efforts on its interaction with proteins involved in actin polymerization, specifically mDia1, VASP, Mena and N-WASP. All four proteins interact with PFN1 through their PLP rich domains. In addition, we also intend to study the interactions of mutant PFN1 with the wild-type PFN1. The rationale for this approach is based on previous work demonstrating that PFN1 might exist as an oligomer in cells⁵²⁻⁵⁴. Alternatively, it is possible that both wild-type PFN1 and mutant may exist within larger complexes involved with actin polymerization and therefore mutant PFN1 may have an influence on the function of WT-PFN1. Axonal cytoskeletal architecture and synapses are critical component of motor neurons and any disruptions can lead to degeneration of these motor neurons.

As indicated, the presence of mutant PFN1 may result in altered binding properties with several of its binding partners. We will investigate the properties of PFN1 binding partners (mDia1, VASP, Mena and N-WASP) using three distinct yet complementary methods: 1) Co-immunoprecipitation (Co-IP) assays, 2) Immunofluorescence of PFN1 transfected cells and 3) Fluorescent Resonance Energy Transfer (FRET) assays.

Co-IP Assays:

Co-IP assays will shed light on whether mutations in PFN1 reduce its affinity for its binding partners. To perform Co-IP assays, mutant PFN1 and wild-type mouse brain and pooled spinal cords extracts from mice at pre-onset to end stage of disease will be analyzed. Immunoprecipitations will be performed using PFN1 antibody followed by Western blotting (Co-IP) to determine the level of bound endogenous proteins mDia1, VASP, Mena and N-WASP and PFN1. Endogenous PFN1 (wild-type) can be detected due to differences in its mobility since human PFN1 run slightly above mouse PFN1. The conditions of this reaction will be similar to those used in determining the binding efficiency of PFN1 to actin and adjusted accordingly. The efficiency of the Co-IP of the each protein listed with either WT or mutant PFN1 will be quantified by western blotting on an Odyssey Infrared Imaging System (Li-Cor). Results from at least 3 independent experiments will be tested for statistical significance using a paired Student's T test.

PFN1 Immunofluorescence:

We will use PFN1 mutant and wild-type with fluorescent tags to investigate the altered interactions of mutant PFN1 with its binding partners by immunofluorescence. Similar to our approach above, either V5-tagged WT or mutant PFN1 will be transfected into primary motor neurons. The cells will be fixed and stained with a V5 antibody (exogenous PFN1) and antibodies for the binding partners described. To examine whether WT-PFN is also sequestered into the mutant PFN1 cellular aggregates, it will be necessary to co-transfect with a WT-PFN1 expression construct with a different epitope tag (e.g. HA-). Differences in the co-localization of WT and mutant PFN1 with its binding partners will be compared.

Cell Imaging and Morphology by FRET Assay:

To further investigate the effect of mutations in PFN1 on its association with interacting proteins in living cells, we will utilize the FRET assay, which allows one to determine the dynamics of protein-protein interactions in situ^{47,55,56}. This approach has been successfully used to investigate actin interactions with different regulatory proteins^{57,58}. Non-transgenic PMNs will be transfected with wild-type or mutant PFN1 fused to the cyan fluorescent protein (CFP). The PFN1-interacting partner (e.g. actin, Mena, etc) fused to the yellow fluorescent protein YFP will be cotransfected. The fluorescence emission of the YFP fluorophore (acceptor) will be measured in response to the excitation of the CFP fluorophore (donor). To correct for the leak-through of donor and the emission of the acceptor after direct excitation, images of the donor-alone and the acceptor-alone control specimens, in addition to the double-label specimens will be acquired. Images will be analyzed using an ImageJ based algorithm^{59,60} to quantify FRET signals. The data generated in FRET assays is qualitative and yields insight into relative wild-type and mutant PFN1 binding affinity, with its binding partners. Mutant and wild-type PFN1 primary motor neuron cultures will be co-transfected with the FRET constructs and images will be acquired with an epifluorescence microscope. The fluorescence intensity of FRET and a control reporter (CFP) will be quantified in >110 cells per condition in at least 3 independent experiments (d=0.5; α=0.05; power=0.95). In order to establish whether the localization of binding is altered, we will additionally quantitate the distribution of PFN1-containing FRET granules (e.g. axons, dendrites, and cell bodies) in motor neurons. As mutant PFN1 forms aggregates in 15-60% of cells, analysis will be separated into aggregate-containing and non-aggregate containing cells. The presence of FRET in cellular aggregates may be observed suggesting that mutant PFN1 can act through a gain-of-function by recruiting its binding partners. If this is the case, our analysis will be adjusted accordingly. Statistical comparisons of the sub-cellular localization of binding will be incorporated into our analysis. As positive control for the FRET experiments, the association of PFN1 with actin will be tested, as we have shown that mutations in PFN1 severely impair their interaction (Wu et al., 2012, FIG. 2). The localization and transport of GFP-tagged mutant and wild type PFN1 will be monitored by live cell imaging using quantitative FRET assays in PMNs. Binding partner constructs with fluorescence tag will be used to transfect cells for FRET studies. Appropriate data fitting and statistical analysis of the fluorescence raw data will be used for the interpretation of results⁴⁷.

In Vivo Studies to Assess PFN1 Mutation Effects on Formation of Aggregates, Actin Polymerization, Cytoskeleton and Synapse:

Finally, in vivo experiments will be performed to analyze hPFN1^{G118V}-actin interaction including hPFN1^{G118V}-actin binding, actin polymerization and ATP level in cervical, thoracic, and lumbar spinal cord tissue, and brain motor cortex. PFN1-actin binding will be quantified by western blotting using anti-PFN1 and anti-actin antibodies. Actin polymerization will be assessed by quantifying the ratio of monomeric G-actin to polymerized hPFN1^{G118V}-actin using Western blot⁶¹. ATP levels will be determined by ATP assay (Abcam). NP-40-insoluble/soluble cell lysate fractionation will be used to quantify insoluble PFN1, TDP-43, and other potential binding partners of PFN1 that may co-exist in insoluble fraction. Finally, we will assess the effects of the PFN1 mutation on motor neuron morphology and the presence of aggregates and PFN1 binding partners at the synapse.

Mutant hPFN1^{G118V} protein is expected to be found in the insoluble fraction in tissue extract from mutant mice. The presence of PFN1 binding partners in the insoluble fraction will be further verified if they co-aggregate with mutant PFN1 using Co-IP and Western blot. Identification of PFN1 binding partners in insoluble fractions and in Co-IP confirm our in vitro data and validate that mutant PFN1 results in formation of aggregates in vivo. The proposed studies will further identify additional proteins present in aggregates found in PFN1 mutant mice. These findings will provide insights into the pathogenic mechanisms of motor neuron and axonal degeneration in ALS due to the PFN1^{G118V} mutation.

### REFERENCES FOR THE EXAMPLES

1. Wu, C. H. et al. Mutations in the profilin 1 gene cause familial amyotrophic lateral sclerosis. Nature 488, 499-503 (2012).
2. Witke, W., Sutherland, J. D., Sharpe, A., Arai, M. & Kwiatkowski, D. J. Profilin I is essential for cell survival and cell division in early mouse development. Proceedings of the National Academy of Sciences of the United States of America 98, 3832-3836 (2001).
3. Ingre, C. et al. A novel phosphorylation site mutation in profilin 1 revealed in a large screen of US, Nordic, and German amyotrophic lateral sclerosis/frontotemporal dementia cohorts. Neurobiology of aging 34, 1708 e1701-1706 (2013).
4. Smith, B. N. et al. Novel mutations support a role for Profilin 1 in the pathogenesis of ALS. Neurobiol Aging (2014).
5. Daoud, H. et al. Mutation analysis of PFN1 in familial amyotrophic lateral sclerosis patients. Neurobiology of aging (2012).
6. Lattante, S., Le Ber, I., Camuzat, A., Brice, A. & Kabashi, E. Mutations in the PFN1 gene are not a common cause in patients with amyotrophic lateral sclerosis and frontotemporal lobar degeneration in France. Neurobiology of aging 34, 1709 e1701-1702 (2013).
7. Tiloca, C. et al. Screening of the PFN1 gene in sporadic amyotrophic lateral sclerosis and in frontotemporal dementia. Neurobiology of aging 34, 1517 e1519-1510 (2013).
8. Al-Chalabi, A. et al. Deletions of the heavy neurofilament subunit tail in amyotrophic lateral sclerosis. Hum Mol Genet 8, 157-164 (1999).
9. Gros-Louis, F. et al. A frameshift deletion in peripherin gene associated with amyotrophic lateral sclerosis. J Biol Chem 279, 45951-45956, doi:10.1074/jbc.M408139200 (2004).
10. Puls, I. et al. Mutant dynactin in motor neuron disease. Nat Genet 33, 455-456, doi:10.1038/ng1123 (2003).
11. Smith, B. N. et al. Exome-wide Rare Variant Analysis Identifies TUBA4A Mutations Associated with Familial ALS. Neuron 84, 324-331 (2014).
12. Rademakers, R. & van Blitterswijk, M. Excess of Rare Damaging TUBA4A Variants Suggests Cytoskeletal Defects in ALS. Neuron 84, 241-243 (2014).
13. Witke, W. et al. In mouse brain profilin I and profilin II associate with regulators of the endocytic pathway and actin assembly. The EMBO journal 17, 967-976 (1998).
14. Mockrin, S. C. & Korn, E. D. Acanthamoeba profilin interacts with G-actin to increase the rate of exchange of actin-bound adenosine 5′-triphosphate. Biochemistry 19, 5359-5362 (1980).
15. Tilney, L. G., Bonder, E. M., Coluccio, L. M. & Mooseker, M. S. Actin from Thyone sperm assembles on only one end of an actin filament: a behavior regulated by profilin. The Journal of cell biology 97, 112-124 (1983).
16. Witke, W. The role of profilin complexes in cell motility and other cellular processes. Trends in cell biology 14, 461-469 (2004).
17. Lefebvre, S. et al. Identification and characterization of a spinal muscular atrophy-determining gene. Cell 80, 155-165 (1995).
18. Johnson, J. O. et al. Exome sequencing reveals VCP mutations as a cause of familial ALS. Neuron 68, 857-864 (2010).
19. Fischer, M. et al. Prion protein (PrP) with amino-proximal deletions restoring susceptibility of PrP knockout mice to scrapie. EMBO J 15, 1255-1264 (1996).
20. Xu, Y. F. et al. Expression of mutant TDP-43 induces neuronal dysfunction in transgenic mice. Mol Neurodegener 6, 73 (2011).
21. Schilling, G. et al. Intranuclear inclusions and neuritic aggregates in transgenic mice expressing a mutant N-terminal fragment of huntingtin. Hum Mol Genet 8, 397-407 (1999).
22. Kiaei, M. et al. Matrix metalloproteinase-9 regulates TNF-alpha and FasL expression in neuronal, glial cells and its absence extends life in a transgenic mouse model of amyotrophic lateral sclerosis. Exp Neurol 205, 74-81 (2007).
23. Esmaeili, M. A., Panahi, M., Yadav, S., Hennings, L. & Kiaei, M. Premature death of TDP-43 (A315T) transgenic mice due to gastrointestinal complications prior to development of full neurological symptoms of amyotrophic lateral sclerosis. Int J Exp Pathol 94, 56-64 (2013).
24. Wegorzewska, I., Bell, S., Cairns, N. J., Miller, T. M. & Baloh, R. H. TDP-43 mutant transgenic mice develop features of ALS and frontotemporal lobar degeneration. Proceedings of the National Academy of Sciences of the United States of America 106, 18809-18814 (2009).
25. Filali, M., Lalonde, R. & Rivest, S. Sensorimotor and cognitive functions in a SOD1(G37R) transgenic mouse model of amyotrophic lateral sclerosis. Behav Brain Res 225, 215-221 (2011).
26. Kiaei, M. et al. Thalidomide and lenalidomide extend survival in a transgenic mouse model of amyotrophic lateral sclerosis. The Journal of neuroscience: the official journal of the Society for Neuroscience 26, 2467-2473 (2006).
27. Vinsant, S. et al. Characterization of early pathogenesis in the SOD1(G93A) mouse model of ALS: part II, results and discussion. Brain Behav 3, 431-457 (2013).
28. Simon, C. M., Jablonka, S., Ruiz, R., Tabares, L. & Sendtner, M. Ciliary neurotrophic factor-induced sprouting preserves motor function in a mouse model of mild spinal muscular atrophy. Hum Mol Genet 19, 973-986 (2010).
29. Shefner, J. M., Cudkowicz, M. & Brown, R. H., Jr. Motor unit number estimation predicts disease onset and survival in a transgenic mouse model of amyotrophic lateral sclerosis. Muscle Nerve 34, 603-607 (2006).
30. Teng, Y. D. et al. Multimodal actions of neural stem cells in a mouse model of ALS: a meta-analysis. Sci Transl Med 4, 165ra164 (2012).
31. Hadano, S. et al. Mice deficient in the Rab5 guanine nucleotide exchange factor ALS2/alsin exhibit age-dependent neurological deficits and altered endosome trafficking. Hum Mol Genet 15, 233-250 (2006).
32. Shefner, J. M. et al. Effect of neurophilin ligands on motor units in mice with SOD1 ALS mutations. Neurology 57, 1857-1861 (2001).
33. Jeanpretre, N. & Kraftsik, R. A program for the computation of power and determination of sample size in hierarchical experimental designs. Computer Methods and Programs in Biomedicine 29, 179-190 (1989).
34. Hassona, M. D. et al. Vascular hypertrophy-associated hypertension of profilin1 transgenic mouse model leads to functional remodeling of peripheral arteries. American journal of physiology. Heart and circulatory physiology 298, H2112-2120 (2010).
35. Moustafa-Bayoumi, M. et al. Vascular hypertrophy and hypertension caused by transgenic overexpression of profilin 1. The Journal of biological chemistry 282, 37632-37639 (2007).
36. Franco, M. C. et al. Nitration of Hsp90 induces cell death. Proceedings of the National Academy of Sciences of the United States of America 110, E1102-1111 (2013).
37. Pedrini, S. et al. ALS-linked mutant SOD1 damages mitochondria by promoting conformational changes in Bcl-2. Hum Mol Genet 19, 2974-2986 (2010).
38. Gould, T. W. et al. Complete dissociation of motor neuron death from motor dysfunction by Bax deletion in a mouse model of ALS. J Neurosci 26, 8774-8786 (2006).
39. Higgins, C. M., Jung, C. & Xu, Z. ALS-associated mutant SOD1G93A causes mitochondrial vacuolation by expansion of the intermembrane space and by involvement of SOD1 aggregation and peroxisomes. BMC Neurosci 4, 16 (2003).
40. Mattiazzi, M. et al. Mutated human SOD1 causes dysfunction of oxidative phosphorylation in mitochondria of transgenic mice. The Journal of biological chemistry 277, 29626-29633 (2002).
41. Gottlieb, E., Armour, S. M. & Thompson, C. B. Mitochondrial respiratory control is lost during growth factor deprivation. Proc Natl Acad Sci USA 99, 12801-12806 (2002).
42. Kawamata, H. et al. Abnormal intracellular calcium signaling and SNARE-dependent exocytosis contributes to SOD1G93A astrocyte-mediated toxicity in amyotrophic lateral sclerosis. J Neurosci 34, 2331-2348 (2014).
43. Frakes, A. E. et al. Microglia induce motor neuron death via the classical NF-kappaB pathway in amyotrophic lateral sclerosis. Neuron 81, 1009-1023 (2014).
44. Re, D. B. et al. Necroptosis drives motor neuron death in models of both sporadic and familial ALS. Neuron 81, 1001-1008 (2014).
45. Taylor, A. R. et al. Astrocyte and muscle-derived secreted factors differentially regulate motoneuron survival. J Neurosci 27, 634-644 (2007).
46. Wu, A. S. et al. Iron porphyrin treatment extends survival in a transgenic animal model of amyotrophic lateral sclerosis. Journal of neurochemistry 85, 142-150 (2003).
47. Ishikawa-Ankerhold, H. C., Ankerhold, R. & Drummen, G. P. Advanced fluorescence microscopy techniques—FRAP, FLIP, FLAP, FRET and FLIM. Molecules 17, 4047-4132 (2012).
48. West, M. et al. The arachidonic acid 5-lipoxygenase inhibitor nordihydroguaiaretic acid inhibits tumor necrosis factor alpha activation of microglia and extends survival of G93A-SOD1 transgenic mice. J Neurochem 91, 133-143 (2004).
49. Hensley, K. et al. Message and protein-level elevation of tumor necrosis factor alpha (TNF alpha) and TNF alpha-modulating cytokines in spinal cords of the G93A-SOD1 mouse model for amyotrophic lateral sclerosis. Neurobiol Dis 14, 74-80 (2003).
50. Suetsugu, S., Miki, H. & Takenawa, T. The essential role of profilin in the assembly of actin for microspike formation. The EMBO journal 17, 6516-6526 (1998).
51. Wills, Z., Marr, L., Zinn, K., Goodman, C. S. & Van Vactor, D. Profilin and the Abl tyrosine kinase are required for motor axon outgrowth in the Drosophila embryo. Neuron 22, 291-299 (1999).
52. Babich, M., Foti, L. R., Wong, L. & Pack, G. R. In vitro translation and computational analyses of human profilin multimers. Proc West Pharmacol Soc 48, 39-43 (2005).
53. Korupolu, R. V. et al. Profilin oligomerization and its effect on poly (L-proline) binding and phosphorylation. International journal of biological macromolecules 45, 265-273 (2009).
54. Bjorkegren-Sjogren, C., Korenbaum, E., Nordberg, P., Lindberg, U. & Karlsson, R. Isolation and characterization of two mutants of human profilin I that do not bind poly(L-proline). FEBS letters 418, 258-264 (1997).
55. Gau, D., Ding, Z., Baty, C. & Roy, P. Fluorescence Resonance Energy Transfer (FRET)-based Detection of Profilin-VASP Interaction. Cellular and molecular bioengineering 4, 1-8 (2011).
56. Pollitt, S. K. et al. A rapid cellular FRET assay of polyglutamine aggregation identifies a novel inhibitor. Neuron 40, 685-694 (2003).
57. Stolting, G. et al. Direct Interaction of CaVbeta with Actin Up-regulates L-type Calcium Currents in HL-1 Cardiomyocytes. J Biol Chem (2014).
58. Fried, S. et al. Triple-color FRET analysis reveals conformational changes in the WIP-WASp actin-regulating complex. Sci Signal 7, ra60 (2014).
59. Wallrabe, H. & Periasamy, A. Imaging protein molecules using FRET and FLIM microscopy. Curr Opin Biotechnol 16, 19-27 (2005).
60. Sun, Y. & Periasamy, A. Additional correction for energy transfer efficiency calculation in filter-based Forster resonance energy transfer microscopy for more accurate results. J Biomed Opt 15, 020513 (2010).
61. Zeng, L. H. et al. Kainate seizures cause acute dendritic injury and actin depolymerization in vivo. The Journal of neuroscience: the official journal of the Society for Neuroscience 27, 11604-11613 (2007).

1. A genetically modified animal comprising at least one exogenous nucleic acid, wherein the exogenous nucleic acid comprises a polynucleotide encoding a human profilin1 protein.

2. The genetically modified animal of claim 1, wherein the polynucleotide encodes a mutated human profilin1 protein.

3. The genetically modified animal of claim 2, wherein the polynucleotide encodes for a mutated human profilin1 protein comprising a mutation selected from the group consisting of C71G, E117G, and G118V relative to SEQ ID NO:1.

4. The genetically modified animal of claim 3, wherein the mutation is G118V.

5. The genetically modified animal of claim 1, wherein the exogenous nucleic acid is operably linked to a mouse prion promoter.

6. The genetically modified animal of claim 1, wherein the human profilin1 protein is overexpressed relative to the endogenous profilin1 protein.

7. The genetically modified animal of claim 1, wherein the human profilin1 protein is expressed in the brain, spinal cord and skeletal muscle.

8. The genetically modified animal of claim 1, wherein the animal develops amyotrophic lateral sclerosis (ALS).

9. (canceled)

10. (canceled)

11. A genetically modified cell, the cell comprising at least one exogenous nucleic acid, wherein the exogenous nucleic acid comprises a polynucleotide encoding a human profilin1 protein.

12. The genetically modified cell of claim 11, wherein the cell is a sperm cell.

13. The genetically modified cell of claim 11, wherein the polynucleotide encodes a mutated human profilin1 protein.

14. The genetically modified cell of claim 13, wherein the polynucleotide encodes for a mutated human profilin1 protein comprising a mutation selected from the group consisting of C71G, E117G, and G118V relative to SEQ ID NO:1.

15. The genetically modified cell of claim 14, wherein the mutation is G118V.

16. The genetically modified cell of claim 11, wherein the exogenous nucleic acid is operably linked to at least a portion of a regulatory region of a mouse prion gene.

17. The genetically modified cell of claim 11, wherein the human profilin1 protein is overexpressed relative to the endogenous profilin1 protein.

18. A method for assessing the therapeutic potential of an agent on an animal, the method comprising:
a) administering an agent to a genetically modified animal comprising at least one exogenous nucleic acid, wherein the exogenous nucleic acid comprises a polynucleotide encoding a human profilin1 protein; and
b) comparing results of a selected parameter to results obtained from a second genetically modified animal which was not administered the agent, wherein the selected parameter is chosen from: weight loss, hindlimb muscle atrophy, histopathology, behavior and premature death.

19. The method of claim 18, wherein the agent is a pharmaceutically active ingredient, a drug, a toxin, or a chemical.

20. The method of claim 18, wherein the method further comprises determining if the agent abates the selected parameter.

21. The method of claim 18, wherein the polynucleotide encodes a mutated human profilin1 protein.

22. The method of claim 21, wherein the polynucleotide encodes for a mutated human profilin1 protein comprising a mutation selected from the group consisting of C71G, E117G, and G118V relative to SEQ ID NO:1.

23.-25. (canceled)
